(19) United States
(12) Patent Application Publication
(10) Pub. No.: US 2015/0197808 A1
(43) Pub. Date: Jul. 16, 2015

(54) CYTOKINES AS PROGNOSTIC MARKERS OF RESPIRATORY-TRACT INFECTION FOLLOWING MAJOR SURGERY

(71) Applicant: The Provost, Fellows and Scholars of the College of the Holy and Undivided Trinity of Queen Elizabe, Dublin (IE)

(72) Inventors: Thomas Ryan, Castleknock (IE); Mary White, Saggart (IE); Owen Ross McManus, Killiney (IE); Dermot Kelleher, Dun Laoghaire (IE); Patrick Stordeur, Dublin (IE); Vincent Young, Dublin (IE)

(21) Appl. No.: 14/498,575

(22) Filed: Sep. 26, 2014

Related U.S. Application Data
(63) Continuation of application No. 13/132,127, filed on Feb. 9, 2012, now abandoned.
(60) Provisional application No. 61/118,702, filed on Dec. 1, 2008.

(30) Foreign Application Priority Data
Dec. 1, 2008 (EP) 08105902

(51) Int. Cl. C12Q 1/68; G06F 19/18

(52) U.S. Cl. CPC C12Q 1/6883; G06F 19/18; C12Q 2600/158; C12Q 2600/112; G06F 19/3431

(57) ABSTRACT

The invention relates to the use of a certain subset of cytokine markers as prognostic variables of infection status in an individual, and especially as prognostic markers of a patients developing severe infection such as pneumonia, and respiratory tract infection following surgery. The subset of cytokine markers consists of the interleukin cytokines IL-2, IL-7, IL-23, IL-27, and IL-10, and Interferon-γ (INFγ) and Tissue Necrosis Factor-α (TNFα). The markers may be employed as individual prognostic variables of infection status, or they may be used in pairs or other combinations. Generally, the abundance of the markers is correlated with infection status by means of an absolute pre-operative value of biomarker abundance, ratio's of pre-operative to post-operative biomarker abundance, or ratio values for pairs of certain biomarkers within the subset. Typically, cytokine abundance is expressed in terms of mRNA copy number wherein the copy numbers are ideally normalised to a house keeping gene and quantification of mRNA copy number is determined by RT-PCR containing reference serial dilutions of cytokine specific cDNA.

The invention relates to a method of estimating risk of a patient developing a respiratory tract infection following major surgery. In particular, the invention relates to a method of a estimating the risk of a patient developing hospital-acquired pneumonia following cardiothoracic surgery.

### BACKGROUND TO THE INVENTION

Pneumonia, and respiratory tract infection after major surgery are commonplace events. Post operative pneumonia, and respiratory tract infection is costly, generates increased requirement for additional medical care, prolongs hospitalisation, and may occasion considerable excess morbidity and mortality. The risk factors for post operative pneumonia, and respiratory tract infection, have been well described, but largely consist of patient demographic factors, co-morbid illness, and the extent of surgery performed, all of which are generally immutable.

Furthermore these known risk factors merely identify a group of patients with a propensity to develop post operative pneumonia, and respiratory tract infection, and will not accurately identify patients who develop pneumonia, and respiratory tract infection, after surgery. Indeed this propensity is so vague that predictive algorithms based on these risk factors lack definition, and knowledge of these factors has not modified surgical practice.

Major surgery is commonplace, with approximately 4 million major abdominal surgeries performed in the US annually. Accordingly post operative pneumonia, and respiratory tract infection, is a major health issue.

Post operative pneumonia, and respiratory tract infection, is generally regarded as a manifestation of the immune compromise which occurs after major surgery. However it is not currently possible to characterise this immune compromised state with an assay or laboratory test that can be performed in a clinical laboratory in an acute hospital setting. Thus the Medical care for post operative patients is delivered by protocol, in a one size fits all approach, with no attempt to individualise patient care.

### STATEMENTS OF INVENTION

The present invention is based on the surprising discovery that gene expression of any one or more of the cytokines IL-2, IL-7, and IL-23 correlate with risk of a patient developing severe infection such as a respiratory tract infection following major surgery such as cardiothoracic surgery. The mRNA copy number of each of these cytokines has been found to significantly decrease within 24 hours of surgery in patients who develop (hospital acquired) severe infection (as compared to those who do not develop hospital acquired severe infection). The invention may therefore be applied to identify patients who are at risk of developing severe infection as a result of major surgery, which allows the specialised treatment of those patients (for example, by immediately placing the patients on a course of antibiotics, or keeping the patient in an ICU, or both).

In one embodiment, an absolute mRNA copy number value of one or more of the cytokines is determined from a biological sample obtained from the patient post-operatively, and preferably within 48, 36 or 24 hours of surgery (i.e. within 48, 36 or 24 hours following the completion of surgery), and this value can be correlated with a reference value to estimate risk of infection. Suitably, all indices of mRNA copy number values are normalised against a housekeeping gene, for example GAPDH or beta-actin. Typically, the biological sample is a peripheral blood mononuclear cell preparation, especially a mononuclear cell preparation obtained from a buffy coat layer of peripheral blood. Thus, in the case where the biological sample is mononuclear cells obtained from a buffy coat layer of a peripheral blood sample, and wherein the mRNA copy number values are normalised against 10 million copies of beta-actin, the following reference values apply:

IL-23: Patients with an IL-23 mRNA copy number greater than or equal to 60684.9 (per 10 million copies of beta-actin) have a low risk of respiratory tract infection. Patients with a lower copy number have a higher risk of respiratory tract infection;

IL-2: Patients with an IL-2 mRNA copy number greater than or equal to 237 (per 10 million copies of beta-actin) have a low risk of respiratory tract infection. Patients with a lower copy number have a higher risk of respiratory tract infection;

IL-7: Patients with an IL-7 mRNA copy number greater than or equal to 476 (per 10 million copies of beta-actin) have a low risk of respiratory tract infection. Patients with a lower copy number have a higher risk of respiratory tract infection.

Preferably, the method of the invention is employed to identify a patient having a high risk of infection in which the cytokine is IL-2, IL-7 or IL-23, and a biological sample from that patient is then assessed for absolute mRNA copy number value for second, different, cytokine selected from the group consisting of more IL-2, IL-7, IL-23, IL-10, IL-27, TNF-α, and Interferon-γ to further focus the risk of infection. Generally, the same biological sample is used for assaying the different cytokines. Suitable combinations of cytokines are described herein and include: IL-23 and IL-7; IL-23 and Interferon-γ; IL-23 and TNF-α. Different cytokines may be combined to focus risk of infection.

In a particularly preferred embodiment of the invention, the absolute mRNA copy number value is converted to a relative value, and the relative value is correlated with a reference value to indicate or provide an estimate of risk of infection. An advantage of providing a relative value for cytokine copy number is that it reduces or excludes variation in the results that may occur due to, for example, use of different housekeeping genes to provide “corrected” copy number values, use of different types of biological samples, differences due to population genetics, etc.

For example, an absolute pre-operative (mRNA copy number) value for a cytokine relative to an absolute post-operative (mRNA copy number) value for the same cytokine may be employed to calculate a relative value, and the relative value may then be correlated with a reference value to indicate risk of infection. In particular, the pre- and post-operative values may be a function of the mRNA copy number, for example the Log 10 of the cytokine mRNA copy number. In one embodiment, if the difference between the post-operative Log 10 value and the pre-operative Log 10 value (i.e. pre-operative Log 10 mRNA copy number minus post-operative Log 10 mRNA copy number) is a positive number, this correlates with a high risk of infection and if the difference is a negative number, this correlates with a low risk of infection (i.e. risk of infection decreases with a decrease in the relative value). However, if will be appreciated that other functions of the copy numbers may be employed to calculate the relative value, in which risk of increases with a decrease in relative value.

The Applicants have also discovered that gene expression of the cytokines IL-10 and IL-27 are similar in patients that develop severe infection and those that do not develop infection—in other words, there is not a clinically significant difference in mRNA copy numbers of these cytokines between these two groups of patients. These cytokines are therefore useful reference cytokines in algorithms for correlating IL-2, IL-7 and IL-23 values with risk of infection. Thus, instead of employing pre- and post-operative values for a specific cytokine as a means of generating a relative value, it is possible to combine a post-operative value for one (or more) of (a) IL-2, IL-7 and IL-23, with a post-operative value for one (or more) of (b) IL-10 and IL-27, and generate a relative value based on a function of the two values. Suitably, the function is the Log 10 of the mRNA copy number for IL-10 and/or IL-27 minus the Log 10 of the mRNA copy number of IL-2, IL-7 and/or IL-23 (or Log 10 of the mRNA copy number of IL-2, IL-7 and/or IL-23 minus Log 10 of the mRNA copy number for IL-10 and/or IL-27). Relative values based on the following algorithms are particularly powerful (where all values are (a) post-operative values, i.e. generated from a sample obtained from the patient within 24 hours following surgery, and (b) Log 10 mRNA copy number):

### 1. [IL-2+IL-7+IL-23]−[IL-10+IL-27] Score A;

### 2. [IL-10−IL-2] Score B; and

### 3. [IL-10+IL-27]−IL-2 Score C.

### 4. [IL-27−IL-2] Score D

Correlation of the relative value with risk of infection may be performed as follows:

If algorithm 1 is employed, risk of infection may be assessed by correlating Score A with FIG. 14A, or 14B, or a scale of 1 to 7, or any other equivalent scale.

If algorithm 2 is employed, risk of infection may be assessed by correlating Score B with FIG. 15A, or 15B, or a scale of −0.5 to 3, or any other equivalent scale.

If algorithm 3 is employed, risk of infection may be assessed by correlating Score C with FIG. 16A, or 16B, or a scale of 1.0 to 5.5, or any other equivalent scale.

If algorithm 4 is employed, risk of infection may be assessed by correlating Score D with FIG. 17A, or 17B, or a scale of −1.5 to 2.0, or any other equivalent scale.

If the above algorithms, the relative value correlates with risk. Thus, for Score A, as the relative value decreases the risk of infection increases. However, if the algorithm 1 is inverted, then as the relative value decreases, the risk ofm infection will also decrease. Likewise, for algorithms 2, 3 and 4. Thus, the relative value may also be calculated by inverting algorithms 1 to 4, i.e. [IL-2−IL-27]=Algorithm 4 inverted.

Generally, when relative (abundance) values are obtained for one or more of IL-2, IL-7 and IL-23, in which the relative value is a function of pre-operative and post-operative values, the pre-operative and post-operative values may be absolute mRNA copy numbers or a function of the mRNA copy number. When copy numbers proper are employed, the copy numbers may be provided as a ratio, and the ratio is typically compared with a reference ratio to provide an assessment of risk. Reference (or cut-off) ratio's for each of IL-2, IL-7 and IL-23 are provided below. However, it will be appreciated that the copy numbers may also be subtracted to provide a difference, and the difference may be compared with a reference difference to assess risk of infection. When a function of mRNA copy numbers is employed, the function is preferably the Log 10 of the copy number (this equates to a Ct value obtained from a PCR process). In this case the (pre-operative Ct minus post-operative Ct) correlates with risk of infection: if ΔCt is a positive number, this correlates with a high risk of infection, if ΔCt, is a negative number, this correlates with a low risk of infection (i.e. risk of infection decreases as delta Ct decreases). It will be appreciated that use of a different function of the copy number, or different algorithms for calculating the relative value, may be employed. For example, if the calculation of delta Ct is inverted, then risk of infection decreases as delta Ct increases.

Thus, in one aspect, the invention provides a method of assessing risk of a patient developing a respiratory tract infection following major surgery, the method comprising the steps:
of assaying biological sample obtained from the patient within 24 hours of the surgery, typically by means of quantitative PCR, to measure an absolute mRNA copy number value for a cytokine selected from the group consisting of IL-2, IL-7 and IL-23;
converting the absolute mRNA copy number value to a relative value by providing a function of the absolute mRNA copy number value and a reference mRNA copy number value; and
correlating the relative value with risk of infection,
   wherein the reference mRNA copy number value is selected from: a pre-operative mRNA copy number value for a corresponding cytokine or a post-operative mRNA copy number value for a cytokine selected from the group consisting of IL-10 and IL-27.

Thus, the method of the invention typically involves the following steps:

1. Providing an absolute mRNA copy number value for IL-2, IL-7 or IL-23 (by quantitative PCR). This may be a copy number, or a function of the copy number, such as a Ct value provided by quantitative PCR.
2. The absolute mRNA copy number value for IL-2, IL-7 or IL-23 is then converted into a relative mRNA copy number value by providing a function of the absolute value and a reference mRNA copy number value.
2a. In one embodiment, the relative value is a function of a pre-operative mRNA copy number value and the absolute (post-operative) mRNA copy number value for IL-2, IL-7 or IL-23. The function may be, for example, a ratio of the pre-operative and post-operative values, or the difference between the pre-operative and post-operative values. The values may be copy numbers, or a function of the copy number (for example the Ct values).
2b. In an alternative embodiment, the relative value is a function of a post-operative mRNA copy value for IL-2, IL-7 or IL-23 (or a composite value for two or three of these cytokines) and a post-operative copy number value for IL-10 or IL-27 (or a composite value for both). The mRNA copy values may be copy number, or a function of the copy number, for example the Log 10 of the copy number. The function may be, for example, a ratio of values, or the difference between the pre-operative and post-operative values.
3. The final step is the step of correlating the relative value with risk of infection:
where the relative value is a function, for example a ratio, of the pre-operative and post-operative copy numbers, then risk of infection can be correlated by comparing the ratio with a cut-off value specific for that cytokine. Thus, where the ratio is greater than the cut-off value, this indicates that the copy numbers for the cytokine have decrease significantly as a result of surgery, and this correlates with a high risk of respiratory tract infection; the higher the ratio, the higher the difference between the pre- and post-operative values, and therefore the higher the risk of infection. Likewise, where the ratio is less than the cut-off value, this indicates that the mRNA copy numbers for the cytokine have not decreased significantly as a result of surgery, and this correlates with a low risk of respiratory tract infection. For IL-2, the reference ratio is 1.5192. For IL-23, the reference ratio is 1.207. Alternatively, the relative value may be the difference of the pre-operative and post-operative Ct values (ΔCt, in which case a positive value for (ΔCt) indicates a risk of high risk of infection, and a negative value for (ΔCt) indicates a low risk of infection.
where the relative value is a function of a post-operative mRNA copy value for IL-2, IL-7 or IL-23 (or a composite value for two or three of these cytokines) and a post-operative mRNA copy number value for IL-10 or IL-27 (or a composite value for both), the method of correlating the relative value with risk of infection involves comparing the relative value with a scale of risk for this algorithm. Thus, referring to FIG. 15A below, the relative value is the difference of the Log 10 of the IL-10 and IL-2 mRNA copy numbers, and the scale is from −0.5 to 3.0, wherein a −0.5 correlates with low risk and 3.0 correlates with high risk.

Thus, the method of the invention involves determining an absolute mRNA copy number value for IL-2, IL7 or IL-23 from a patient within 24 hours of surgery, conversion of the absolute value to a relative value by providing either (a) a function of pre-operative to post-operative absolute values for a single cytokine, or (b) a function of post-operative absolute value for a first cytokine or combination of cytokines (IL-2, IL-7, or IL-23, or a combination thereof) and a post-operative absolute value for a second cytokine of combination of cytokines (IL-10 and IL-27, or both combined), and correlating the relative value with risk of infection.

The process of the invention generally involves providing corrected mRNA copy numbers, in other words, mRNA copy numbers normalised to a suitable housekeeping gene. The housekeeping gene employed in the examples herein is beta-actin, however other it will be appreciated that other housekeeping genes may be employed. In cases where relative values for IL-2, IL-7 or IL-23 are provided, the risk of infection will not differ significantly if difference housekeeping genes are employed, or if different sources of biological sample are employed.

Thus, the present invention provides method of method for assessing risk of a patient developing a respiratory tract infection, such as pneumonia, as a result of major surgery, for example, cardiothoracic surgery. The term “risk of infection” as used herein generally means a risk of hospital acquired infection. Generally, the cytokines are determined from a mononuclear cell preparation, generally one obtained from a peripheral blood sample from the patient, preferably from the buffy coat layer of such a peripheral blood sample. The method of determining mRNA copy number values preferably employs quantitative polymerase chain reaction (PCR), ideally quantitative real-time PCR. The mRNA copy number value may be mRNA copy number proper, or a function of mRNA copy number, for example the Log 10 of the copy number, or a Ct value obtained from a PCR process.

### BRIEF DESCRIPTION OF THE FIGURES

FIG. 1a: Restriction map and multiple cloning sites for pDNR-LIB vector

FIG. 1b: Map of the pDNR-LIB vector MCS, multiple cloning site. The IL2 cDNA insert replaces the stuffer fragment. Unique restriction sites are shown in bold.

FIG. 2: Restriction map and multiple cloning sites for pCMV-SPORT6 vector

FIG. 3: DNA gel Single Digest gel with EcoR I and Xba I for IL2 and IL7 respectively. 1% agarose DNA Gel: Lane 1 contains a 1 kb ladder. Lane 2 contains linear plasmid IL2 DNA following single restriction enzyme digestion with EcoRI. Lane 3 contains linear plasmid IL7 DNA following single restriction enzyme with XbaI.

FIG. 4: DNA gel Double Digest gel with EcoR I and HIND III for IL2 and EcoR I and Xba I IL7 respectively. 1% agarose DNA Gel: Lane 1 contains a 1 kb ladder. Lane 2 contains linearised plasmid IL2 DNA following double restriction enzyme digestion with EcoRI and HIND III. Lane 3 contains linear plasmid IL7 DNA following double restriction enzyme digestion with XbaI and EcoRI.

FIG. 5: Absorbance Spectrum of plasmid DNA.

FIG. 6a: Standard Curve for IL2. The slope of the standard curve can be used to determine the exponential amplification a efficiency of the QRT-PCR reaction. A slope between −3.2 and −3.6 is an acceptable efficiency. The ideal QRT-PCR reaction has an efficiency of 1.0092 and amplification of 2.0092, this corresponds to a slope of −3.3.

FIG. 6b: Standard Curve for IL7. The slope of the standard curve can be used to determine the exponential amplification a efficiency of the QRT-PCR reaction. A slope between −3.2 and −3.6 is an acceptable efficiency. The ideal QRT-PCR reaction has an efficiency of 1.0092 and amplification of 2.0092, this corresponds to a slope of −3.3.

FIG. 6c: Standard Curve for the house keeping gene β-actin. The slope of the standard curve can be used to determine the exponential amplification a efficiency of the QRT-PCR reaction. A slope between −3.2 and −3.6 is an acceptable efficiency. The ideal QRT-PCR reaction has an efficiency of 1.0092 and amplification of 2.0092, this corresponds to a slope of −3.3.

FIG. 7a: Oneway Analysis of delta Ct IL-2 day 0-1 By Patient Group. There are two patient groups. One developed Respiratory infection and the other group did not develop infection. The delta Ct refers to the Pre operative PCR-Ct value for IL-2—the Post Operative value. Thus a positive value actually represents a decrease in Ct. Day 0 refers to pre operative values, day 1 to post operative values.

FIG. 7b: Logistic Fit of Patient Group By delta ct IL-2 day 0-1. The delta Ct refers to the Pre operative PCR-Ct value for IL-2—the Post Operative value. Thus a positive value actually represents a decrease in Ct. Day 0 refers to pre operative values, day 1 to post operative values.

FIG. 7c: Contingency Analysis of decrease IL-2 By Patient Group Mosaic Plot. This figure displays the frequency of the occurrence of respiratory tract infection after surgery in relation to a categoric change in PCR-Ct value, which was either an increase or a decrease.

FIG. 7d: Oneway Analysis of IL 2 Day 1 Post op By Patient Group. The occurrence of post operative pneumonia can be related to gene transcription data for single cytokines. Thus IL-2 mRNA values are lower on the first day after surgery in patients who subsequently develop post operative pneumonia. In this analysis IL-2 is expressed as a corrected Log base 10 copy number.

FIG. 8a: Oneway Analysis of delta Ct IL-7 day 0-1 By Patient Group. There are two patient groups. One developed Respiratory infection and the other group did not develop infection. The delta Ct refers to the Pre operative PCR-Ct value for IL-7—the Post Operative value. Thus a positive value actually represents a decrease in Ct. Day 0 refers to pre operative values, day 1 to post operative values.

FIG. 8b: Logistic Fit of Patient Group By delta ct IL-7 day 0-1. The delta Ct refers to the Pre operative PCR-Ct value for IL-7—the Post Operative value. Thus a positive value actually represents a decrease in Ct. Day 0 refers to pre operative values, day 1 to post operative values.

FIG. 8c: Contingency Analysis of Decrease IL-7 By Patient Group Mosaic Plot. This figure displays the frequency of the occurrence of respiratory tract infection after surgery in relation to a categoric change in PCR-Ct value, which was either an increase or a decrease.

FIG. 8d: Oneway Analysis of Day 1 IL-7 Post op By Patient Group. The occurrence of post operative pneumonia can be related to gene transcription data for single cytokines. Thus IL-7 mRNA values are lower on the first day after surgery in patients who subsequently develop post operative pneumonia. In this analysis IL-7 is expressed as a corrected Log base 10 copy number.

FIG. 9a: Oneway Analysis of delta Ct IL-23 day 0-1 By Patient Group. There are two patient groups. One developed Respiratory infection and the other group did not develop infection. The delta Ct refers to the Pre operative PCR-Ct value for IL-23—the Post Operative value. Thus a positive value actually represents a decrease in Ct. Day 0 refers to pre operative values, day 1 to post operative values.

FIG. 9b: Logistic Fit of Patient Group By delta ct IL-23 day 0-1. The delta Ct refers to the Pre operative PCR-Ct value for IL-23—the Post Operative value. Thus a positive value actually represents a decrease in Ct. Day 0 refers to pre operative values, day 1 to post operative values.

FIG. 9c: Contingency Analysis of Decrease IL-23 By Patient Group Mosaic Plot. This figure displays the frequency of the occurrence of respiratory tract infection after surgery in relation to a categoric change in PCR-Ct value, which was either an increase or a decrease.

FIG. 9d: Oneway Analysis of Day 1 IL-23 Post op By Patient Group. The occurrence of post operative pneumonia can be related to gene transcription data for single cytokines Thus IL-23 mRNA values are lower on the first day after surgery in patients who subsequently develop post operative pneumonia. In this analysis IL-23 is expressed as a corrected Log base 10 copy number.

FIG. 10a: Oneway Analysis of delta Ct TNFα day 0-1 By Patient Group. There are two patient groups. One developed Respiratory infection and the other group did not develop infection. The delta Ct refers to the Pre operative PCR-Ct value for TNFα—the Post Operative value. Thus a positive value actually represents a decrease in Ct. Day 0 refers to pre operative values, day 1 to post operative values.

FIG. 10b: Logistic Fit of Patient Group By delta Ct TNF day 0-1. The delta Ct refers to the Pre operative PCR-Ct value for TNFα—the Post Operative value. Thus a positive value actually represents a decrease in Ct. Day 0 refers to pre operative values, day 1 to post operative values.

FIG. 10c: Contingency Analysis of Decrease TNF By Patient Group Mosaic Plot. This figure displays the frequency of the occurrence of respiratory tract infection after surgery in relation to a categoric change in PCR-Ct value, which was either an increase or a decrease.

FIG. 11: Contingency Analysis of Combined IL-2 and IL-23 By Patient Group Mosaic Plot. This figure displays the frequency of the occurrence of respiratory tract infection after surgery in relation to a categoric change in PCR-Ct value, which was either an increase or a decrease.

FIG. 12: Oneway Analysis of Day 1 IL-10 Post op By Patient Group. The occurrence of post operative pneumonia can be related to gene transcription data for single cytokines. Thus IL-10 mRNA values are similar on the first day after surgery in patients who subsequently develop post operative pneumonia to patients who do not develop pneumonia. In this analysis IL-10 is expressed as a corrected Log base 10 copy number. As IL-10 is similar in patients who develop pneumonia and those who do not develop pneumonia and thus IL-10 serves as a useful reference cytokine in subsequent algorithms.

FIG. 13: Oneway Analysis of Day 1 IL-27 Post op By Patient Group. The occurrence of post operative pneumonia can be related to gene transcription data for single cytokines. Thus IL-27 mRNA values are similar on the first day after surgery in patients who subsequently develop post operative pneumonia to patients who do not develop pneumonia. In this analysis IL-10 is expressed as a corrected Log base 10 copy number. As IL-27 is similar in patients who develop pneumonia and those who do not develop pneumonia and thus IL-27 serves as a useful reference cytokine in subsequent algorithms.

FIG. 14a: Oneway Analysis of Score A By Patient Group. These cytokines, IL-2, IL-7, IL-10, IL-23 and IL-27, can be summated into a combined score by adding the Log base 10 corrected copy numbers of IL-2, IL-7 and IL-23, and subtracting the log base 10 corrected copy number for IL-10 and IL-27. The Score, Score A, which results for this combination of cytokines is significantly lower in patients who subsequently develop pneumonia.

FIG. 14b: Logistic Fit of Patient Group By Score A. In this analysis increasing score, is associated with a decrease in risk for developing post operative pneumonia. Thus for every unit increase in score the risk of developing pneumonia is multiplied by 0.4165, and the risk of developing pneumonia over the range of the score decreases as the score increases, with the highest score associated with a 0.007 times risk of developing pneumonia compared with the lowest score. Conversely for each unit decrease in score the risk of pneumonia increases by 2.4, with a cumulative increased risk from highest to lowest score of 131 fold.

FIG. 15a: Oneway Analysis of Score B By Patient Group. An alternative algorithm involves the difference in log base 10 corrected copy numbers of IL-10 minus those for IL-2. This score is higher in patients who develop pneumonia after surgery.2.

FIG. 15b: Logistic Fit of Patient Group By Score B. In this analysis increasing score, in this case score B is associated with an increase in risk for developing post operative pneumonia. Thus for every unit increase in score the risk of developing pneumonia is multiplied by 5.96, and the risk of developing pneumonia over the range of the score decreases as the score increases, with the highest score associated with a 263 times risk of developing pneumonia compared with the lowest score.

FIG. 16a: Oneway Analysis of Score C By Patient Group. An alternative algorithm involves the difference in log base 10 corrected copy numbers of IL-10 plus those for IL-27 minus those for IL-2. This score is higher in patients who develop pneumonia after surgery.

FIG. 16b: Logistic Fit of Patient Group By Score C. In this analysis increasing score, in this case score C is associated with a decrease in risk for developing post operative pneumonia.

Thus for every unit increase in score the risk of developing pneumonia is multiplied by 2.5, and the risk of developing pneumonia over the range of the score decreases as the score increases, with the highest score associated with a 59.5 times risk of developing pneumonia compared with the lowest score.

FIG. 17a: Oneway Analysis of Score D By Patient Group. An alternative algorithm involves the difference in log base 10 corrected copy numbers of IL-27 minus those for IL-2. This score is higher in patients who develop pneumonia after surgery.

FIG. 17b: Logistic Fit of Patient Group By Score D. In this analysis increasing score, in this case score D is associated with a decrease in risk for developing post operative pneumonia. Thus for every unit increase in score the risk of developing pneumonia is multiplied by 4.2, and the risk of developing pneumonia over the range of the score decreases as the score increases, with the highest score associated with a 53 times risk of developing pneumonia compared with the lowest score.

### DETAILED DESCRIPTION OF THE INVENTION

Broadly, the invention relates to the use of a certain subset of cytokine markers as prognostic variables of infection status in an individual, and especially as prognostic markers of a patient developing severe infection such as pneumonia, and respiratory tract infection following surgery. The subset of cytokine markers consists of the interleukin cytokines IL-2, IL-7, IL-23, IL-27, and IL-10, and Interferon-γ (INFγ) and Tissue Necrosis Factor-α (TNFα). The markers may be employed as individual prognostic variables of infection status, or they may be used in pairs or other combinations. The invention may be employed to correlate absolute cytokine mRNA copy number values with risk of infection, and relative cytokine mRNA copy number values with risk of infection. Generally, the abundance of the markers is correlated with expression status by means of an absolute value of biomarker abundance, relative biomarker abundance values, or ratio values for pairs of certain biomarkers within the subset.

According to the invention, there is provided a method of assessing infection status in an individual comprising a step of assaying a biological sample from the individual to determine an abundance of a biomarker selected from a sub-group consisting of IL-2, IL-7, IL-23, IL-27, IL-10, INFγ and TNFα and correlating the abundance of the biomarker with infection status.

The term infection status should be taken to include determining the likelihood of a severe infection developing, especially following surgery, the likelihood of an infection being present, or the likelihood that the infection will develop. In a particularly preferred embodiment of the invention, the term “infection status” should be taken to mean the risk of the patient developing severe (i.e. respiratory tract) infection following surgery, especially major surgery. Examples of major surgery include cardio-thoracic surgery, abdominal surgery, major vascular surgery, neurosurgery, head and neck surgery, major urological surgery, major gynaecologic resection, and trauma surgery.

Ventilator associated pneumonia is an important example of infection which may occur after major surgery, or complicating a medical illness or in trauma patients, and is an example of respiratory tract infection to which this invention would be relevant.

Thus the methods of the invention are useful for informing a clinician as to the likelihood of a patient having or developing an infection, and particularly the likelihood of the patient developing severe infection such as pneumonia or respiratory tract infection, following surgery. Thus, the prognostic methods of the invention allow a clinician to identify those patients who are at risk of developing severe infection in response to surgery, and initiate clinical intervention to prevent or treat such infection.

In this specification, the term “severe infection” should be taken to mean an infection which required therapy with antibiotics, or prolonged hospital stay for therapy of an infection, or caused an episode of sepsis. In this context sepsis refers to an infection, which causes bodily organ failure such as shock, respiratory failure, renal failure, coagulation abnormality and or encephalopathy. Examples of severe infection include pneumonia, septicaemia, cellulitis and wound infection, urinary tract infection, and infection of an indwelling device such as a central line or prosthesis.

The term “biological sample” may be any sample obtained from an individual such as, for example, blood, serum, saliva, urine, cerebrospinal fluid, tissue, cells, etc. In a preferred embodiment of the invention, the sample will be a lymphocyte preparation such as lymphocytes contained in the buffy coat layer of a peripheral blood sample. In many cases, the individual will be a person with an established infection, or a person at risk of developing an infection, such as a patient who is immunocompromised due to disease, surgery or other factors. In other cases, the individual may be a person known to have an infection, or severe sepsis, and who is under going a therapeutic treatment regime, in which case the method of the invention may be employed to monitor the effectiveness of the treatment.

Biomarker abundance is provided in the form of absolute biomarker abundance values, relative biomarker abundance values, and changes of pre-operative and post-operative biomarker abundance values. Further, the invention employs both single biomarkers and combinations of biomarkers. The step of correlating biomarker abundance values with infection status involves simple algorithms, the details of which are provided below and the use of which is determined by the biomarker(s) chosen, and the nature of the biomarker abundance value. For example, one or more of the biomarkers may be employed as prognostic variables, and the information provided herein provides guidance on which biomarkers and biomarker combinations provide the optimum predictive power. For example, in one embodiment, the biomarkers IL-2 and IL-23 may be used as single prognostic variables of infection status, including variables of risk of severe infection following surgery. Otherwise, the following combinations may for example be employed: IL-2 and IL-23; IL-23 and IL7; IL-23 and INFγ; and IL23 and TNFα.

Once determined, the determined abundance values for the/each biomarker may be correlated with infection status. In one embodiment, the abundance of a biomarker may be taken in the form of an absolute value which is then compared with a reference value to provide an indication of infection status. Methods of determining absolute values for biomarker abundance will be known to those skilled in the art, and the invention is not limited to any particular method. In the present Application, the preferred method of determining absolute abundance values is determining mRNA copy number. The levels of the prognostic variables of the present invention present in biological samples, especially in patients with compromised immunity, tend to be very low. Accordingly, the method employed in assaying a biological sample for the levels of these cytokines is required to be extremely sensitive. For example, expression level may be quantified by absolute quantification of mRNA copy number, wherein the copy numbers are ideally normalised to a house keeping gene such as, for example, GAPDH or b-Actin. Other suitable housekeeper genes will be known to those skilled in the art. Typically, absolute quantification of mRNA copy number is determined by quantitative PCR (for example, RT-PCR) containing reference serial dilutions of cytokine specific cDNA.

Thus, post-operative patients at have low IL-23 mRNA copy number have a greater risk of developing severe infection, and post-operative patients that have high IL-23 mRNA copy number have a lower risk of developing severe infection. Thus, where abundance is determined in terms of mRNA copy number, post-operative patients patients that have an IL-23 copy number of greater than or equal to 60684.9 are regarded as having a low risk of developing severe infection in response to surgery, whereas patients having a copy number less than this number will be at a greater risk of developing severe infection following surgery. Thus, the abundance of IL-23 correlates with risk of severe infection.

In one embodiment, the method involves employing IL-23 mRNA copy number values to identify a cohort of patients having low IL-23 mRNA copy number values, and then further stratifying the cohort according to mRNA copy number values of a second, different, cytokine, suitably selected from the group consisting of IL-7, and INFγ. Thus, in patients with low IL-23 mRNA copy number values (i.e. a mRNA copy number of less than 60684.9, or an equivalent thereof):
a low IL-7 abundance (11-7 mRNA copy number less than 582.9, or an equivalent thereof) correlates with a high risk of infection;
a low INFγ abundance (mRNA copy number of less than 101, or an equivalent thereof) correlates with a high risk of infection

As will be appreciated, the reference values provided above are determined by a number of factors including the protocol employed, and the choice of housekeeping gene. Thus, use of an alternative protocol, such as for example use of a different house keeping gene, would result in different, equivalent, reference values being employed.

In an alternative embodiment of the invention, biomarker abundance is provided in the form of a function of pre-operative cytokine mRNA copy number value to post-operative cytokine mRNA copy number value. In one embodiment, the function is a ratio of the pre-operative value to the post-operative value (pre-operative value divided by post-operative value). Thus, for example, in terms of IL-23, a high ratio (a ratio of greater than 1.207 (i.e. 1.3 or 1.4), or an equivalent thereof expressed as a different function of pre-operative to post-operative value) correlates with a high risk of developing severe infection.

Pre-operative generally means an abundance value of a given biomarker as determined from a biological sample obtained from a patient within one week prior to proposed surgery, and ideally one, two, or three prior to the start of surgery. Likewise, post-operative abundance value should be understood to mean an abundance value of a given biomarker as determined from a biological sample obtained from a patient within one, two, or three days following the completion of surgery.

Thus, in one aspect, the invention provides a method of assessing the risk of a patient developing severe infection following surgery, the method comprising a step of determining a pre-operative abundance value and a post-operative abundance value for a biomarker selected from the group consisting of IL-2, IL-7, and IL-23, and correlating the change in the pre-operative and post-operative values with risk of severe infection.

Suitably, the change in pre-operative abundance to post-operative abundance is provided as a ratio (pre-operative divided by post-operative values). However, the change in abundance may be expressed as a different function of pre-operative and post-operative values.

Typically, the biomarker is selected from the group consisting of IL-2, and IL-23.

In an embodiment in which the primary the biomarker is IL-2, a high ratio of pre-operative mRNA copy number value to post-operative cytokine mRNA copy number (a ratio greater than 1.5192, or an equivalent thereof) correlates with a high risk of severe infection following surgery, and a low ratio (a ratio less than 1.5192, or an equivalent thereof) correlates with a low risk of developing severe infection. Typically, the method employs IL-2 and IL-23 as biomarkers, wherein patients with a high IL-2 pre-operative to post-operative ratio, and a very high IL-23 pre-operative to post-operative ratio (a ratio greater than 1.4115, or an equivalent thereof) have a very high risk of developing severe infection in response to surgery.

In another embodiment in which the primary biomarker is IL-23, a high ratio of pre-operative cytokine mRNA copy number value to post-operative cytokine mRNA copy number value (a ratio greater than 1.207, or an equivalent thereof) correlates with a high risk of severe infection following surgery, and a low ratio (a ratio less than 1.207, or an equivalent thereof) correlates with a low risk of developing severe infection. In another embodiment, the method employs IL-23 and IL-7 as biomarkers, wherein patients with a high pre-operative to post-operative IL-23 ratio and a high IL-7 pre-operative to post-operative ratio (a ratio greater than 1.106) have a very high risk of developing severe infection in response to surgery. In yet another embodiment, the method employs IL-23 and INFγ as biomarkers, wherein patients with a high pre-operative to post-operative IL-23 ratio (a ratio greater than 1.207, or an equivalent thereof) and a high INFγ pre-operative to post-operative ratio (a ratio greater than 1.106, or an equivalent thereof) have a very high risk of developing severe infection in response to surgery.

In another embodiment of the invention, biomarker abundance is provided in the form of an IL-23/IL-27 ratio (of mRNA copy number values), wherein a high ratio (a ratio of greater than or equal to 251) correlates with a low risk of pneumonia, and wherein a low ratio (a ratio of less than 251) correlates with a greater risk of developing pneumonia (as compared with the low-risk group). Typically, the cohort identified as having a low IL-23/IL-27 ratio and who have a low INFg/IL-10 ratio (a ratio of less than 0.0171) have a high risk of developing severe infection in response to infection.

### Characterisation of Patients Groups

Patients who were scheduled for elective thoracic surgery gave informed consent, and blood was drawn for subsequent assays on the day before surgery, and on the first and fifth day after surgery. Thoracic surgery patients are an ideal group, as these patients have no infections prior to surgery, and the surgery involves a relatively sterile body cavity. Patients with obvious cause of immune compromise, such as HIV/AIDS, neutropoenia, and or high steroid dosage, were excluded from this study.

### Description of Methods of Determining Copy Number

In order to provide internal quality control for the PCR process and so as to permit comparison between PCR runs, each run was performed with a cytokine specific standard or reference serial dilution of cDNA. These reference or standard cytokine specific cDNA were prepared for all cytokines, including IL-2, IL-7, IL-10, IL-23, IL-27, TNFα and Interferon gamma. As an additional control, all indices of gene expression were normalised to a house keeping gene, in this instance β-actin.

There are two methods for producing these cytokine specific references or standard serial dilutions of cDNA. We have incorporated both methods. IL10, IL23, IL27, TNFα, β-Actin and Interferon-γ standards were prepared by Dr Patrick Stordeur as per Stordeur et al J. Immunological Methods 259(2002) 55-64. Alternatively cDNA references may be prepared from a commercially available plasmid; as is the case with the IL-2 and IL-7 cDNA standards. The specific cytokine sequence is integrated within the vector; and the serial dilution of the standard curve for IL-2 and IL-7 is outlined below.

### Preparation of the IL2 Standard

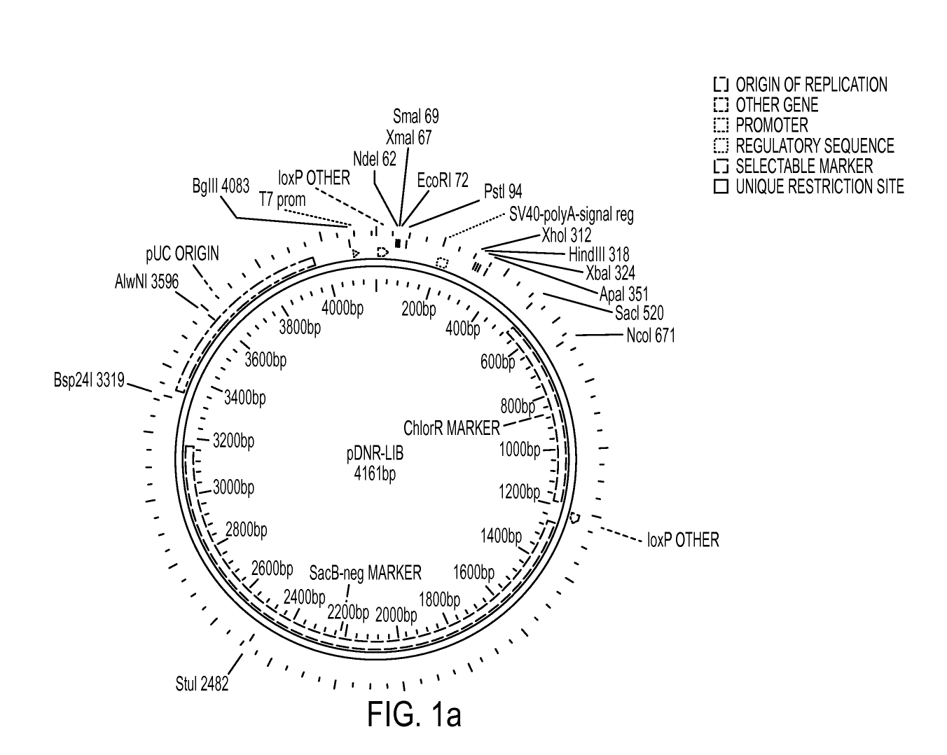

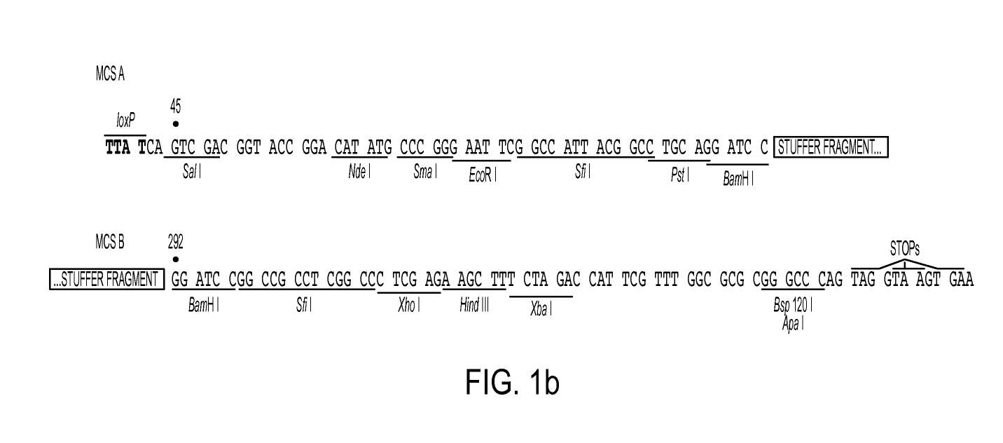

IL2 plasmid was purchased from Open Biosystems (MHS1011-98053730 Human MGC Verified FL cDNA IRAU). It consisted of an 894 bp cDNA clone inserted into a 4.161 kb pDNR-LIB vector. This is illustrated in FIG. 1.

### Preparation of the IL7 Standard

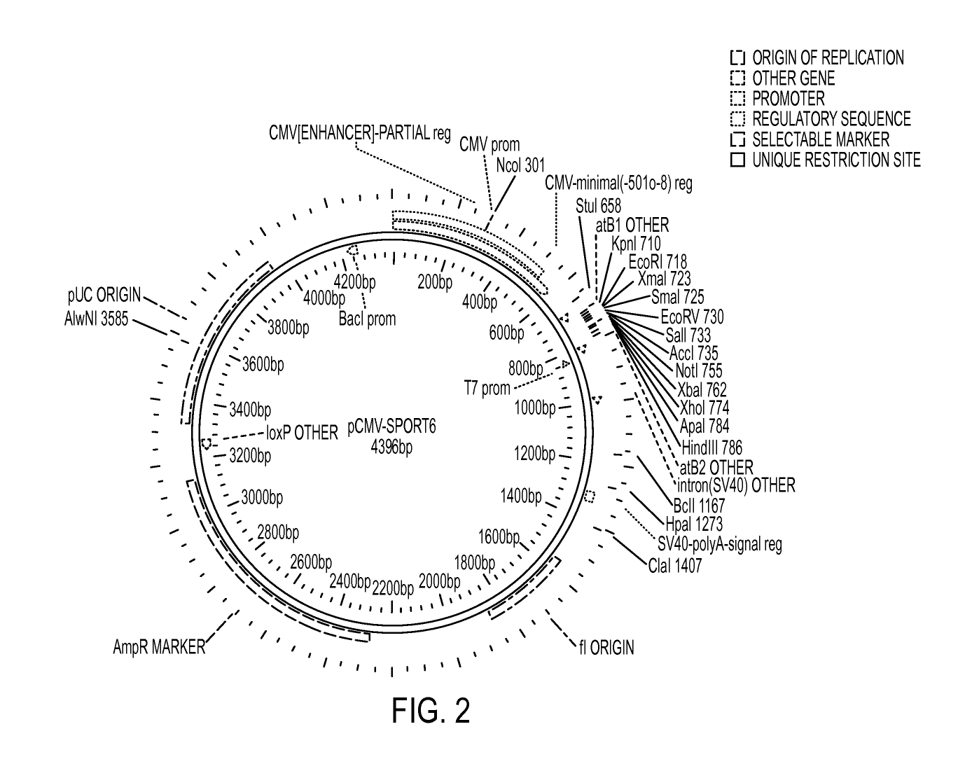

IL7 plasmid was purchased from Open Biosystems (MHS1010-9205095 Human MGC Verified FL cDNA IRAT). It consisted of an 2125 bp cDNA clone inserted into a 4396 bp pCMV-Sport6 vector. This is illustrated in FIG. 2

### Plasmid Culture Conditions

An E. coli culture harbouring the pDNR-LIB vector containing the IL-2 gene was streaked onto a chloramphenicol (25 m/ml) containing LB agar plate and incubated at 37° C. overnight. A similar E. coli culture harbouring the pCMV-SPORT6 vector containing the IL-7 gene was streaked onto an AGAR plate containing ampicillin (100 n/ml). A single colony was isolated from each plate and streaked onto another plate. A well-isolated colony from this second plate was then used to inoculate a liquid L-broth culture grown overnight at 37° C. for each plasmid. These steps ensure the isolation of a clone of a single bacterium.

### Purification of DNA

The Fast Ion Plasmid Midi kit Fast Ion™ (Cat No YP125/YPM10), was used to purify plasmid DNA from 100 ml overnight cultures of E. coli according to the manufacturers' instructions. Bacteria were lysed and the cleared lysate is passed through a cation-exchange column, which binds the re-natured plasmid DNA. The column with bound DNA was washed repeatedly and the DNA is eluted in a high-salt buffer. The DNA is then further purified and desalted by precipitation with isopropanol and resuspended in ddH₂O. Purified plasmid DNA was visualized on a 1% agarose gel as detailed below.

### Agarose Gel Electrophoresis

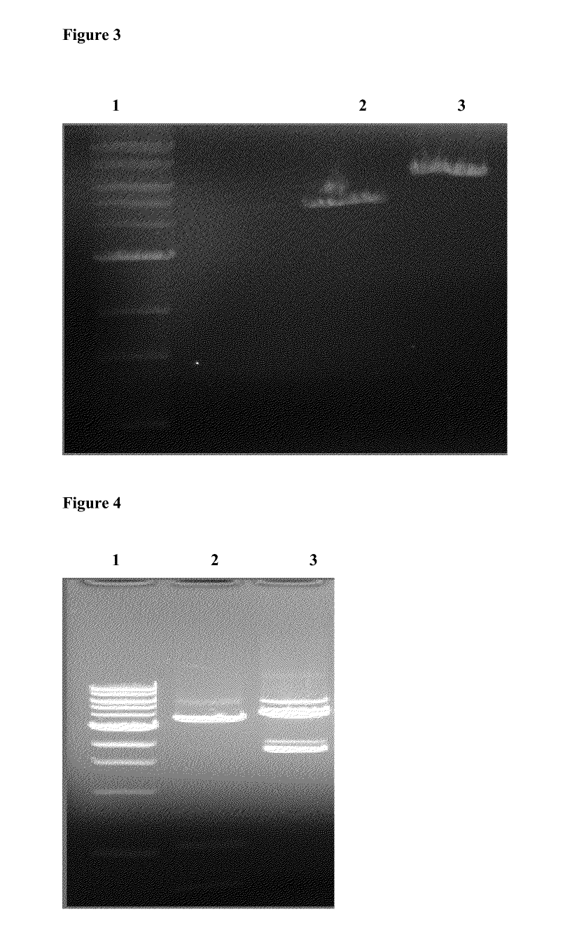

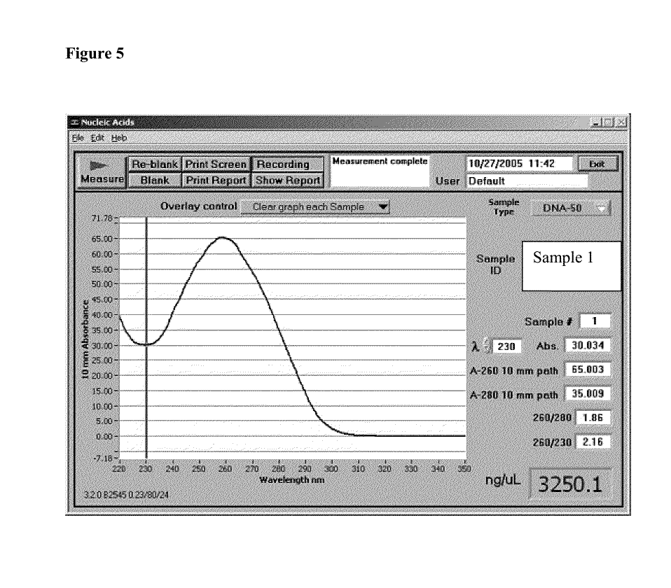

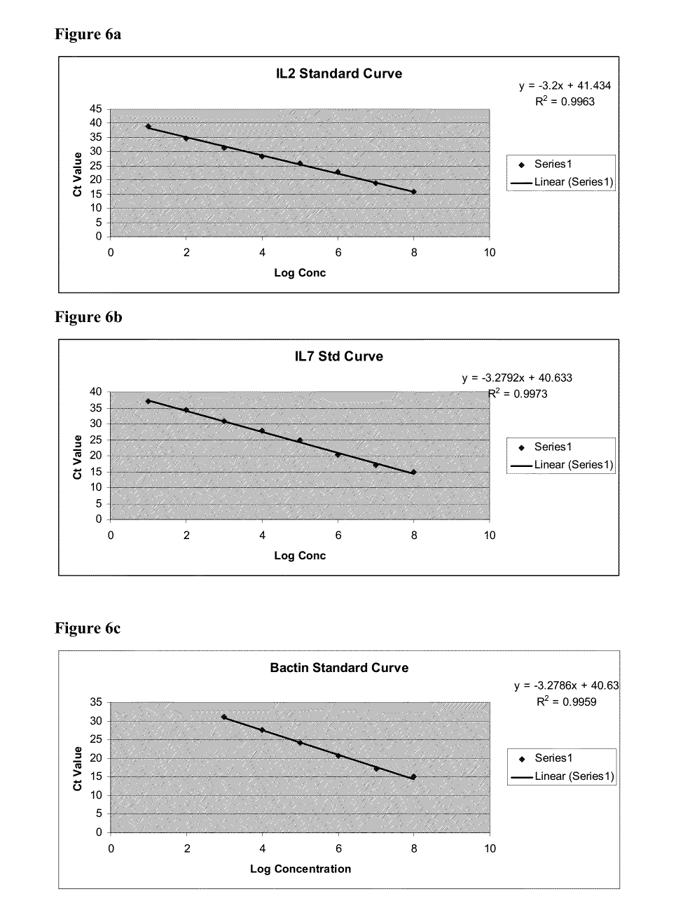

DNA samples were visualised following separation on a 1% agarose gel. Briefly, for a 1% gel, agarose (1 g) was added to 100 ml of 0.5×TBE buffer (44.5 mM tris borate, pH 8.3, 1 mM EDTA) and heated to 100° C. to dissolve the agarose. Ethidium bromide was added to a final concentration of 1 μg/ml and the molten gel was poured into a gel mould and allowed to set. DNA samples were prepared by adding an appropriate volume of 5× sample loading buffer (25 mM tris pH 7.6, 30% (v/v) glycerol, 0.125% (w/v) bromophenol blue) and these samples were electrophoresed through the gel at 135 V for 45 min in 0.5×TBE buffer. The separated DNA fragments were photographed while illuminated under UV light (FIG. 3).

### Restriction Endonuclease Digestion of DNA

All restriction digests were carried out using enzymes supplied by New England Biolabs (NEB) according to the manufacturers' instructions. Briefly, 0.1-2 μg of purified DNA was incubated with 10-20U of restriction enzyme in the appropriate NEB buffer for 2 h at the appropriate temperature. Digests with double enzymes were carried out in the recommended double digest buffer, in which all enzymes had 100% activity (FIG. 4).

### Clone Verification

The IL2 and IL7 clone was end sequenced by MWG-Biotech, Ebersberg, Germany. This was verified against the GeneBank sequence for IL2 and IL7 using BLAST.

DNA was quantified and qualified using the Nanodrop® ND 8000 (220-750 nm) full spectrum spectrophotometer. Briefly a 1 μl sample of DNA was placed on the measuring pedestal. The pedestal is actually the end of a fibre optic cable (receiving fibres). A second set of fiber optic cables (the source fiber) are then brought in contact with the liquid sample, causing the liquid to bridge the gaps between the fiber optic ends. A pulsed xenon flash lamp provides the light source and a spectrometer using a linear CCD array is used to analyse the light that passes through the samples. Absorbance measurements, measure any molecules absorbing at a specific wavelength. Nucleotides, RNA, ssDNA and dsDNA all absorb at 260 nm and contribute to the overall absorbance. The ratio of absorbance at 260 nm and 280 nm is used to assess the purity of DNA and RNA. A ratio of ˜1.8 is accepted as “pure” for DNA; a ratio ˜2.0 is accepted as “pure” for RNA. If the ratio is lower it indicates the presence of contaminants. The 260/230 ratio is used as a secondary measure of nucleic acid purity. The 260/230 values for “pure” nucleic acid are expected to be in the range of 2.0-2.2. The absorbance spectrum in FIG. 2.5 indicates a DNA concentration of 3250.1 ng/μl, with a 260-280 ratio of 1.86 and a 260-230 ratio of 2.16. The absorbance spectrum for IL2 and IL7 are presented in table 1

TABLE 1
Results from absorbance spectrum λ 230

| | A260 | A280 | | | Conc |
|---|---|---|---|---|---|
| Sample | 10 mm path | 10 mm path | 260/280 | 260/230 | ng/μl |
| IL2 | 10.314 | 5.548 | 1.86 | 2.05 | 515.7 |
| IL7 | 67.852 | 37.271 | 1.83 | 2.08 | 3365.6 |

Determining the Volume of Plasmid DNA Corresponding to Copy Numbers of Target Nucleic Acid Sequences, i.e. Creating a Standard Curve with a Plasmid DNA Template.

To create a Standard Curve with Plasmid DNA template from which both, the cloned IL2 and IL7 sequence is present in 10*8 to 10*0 copies correspondingly. This standard curve is utilised to calculate absolute copy numbers of IL2 and IL7 mRNA in patient samples. Our quantitative real time PCR reactions are set up such that 1.5 μl of plasmid DNA is pipetted into each QRT-PCR reaction.

### IL2 Standard Curve

The stock of IL2 plasmid DNA was determined to be 515.7 ng/μl by spectrophotometric analysis. The vector size for pDNR-LIB is 4161 bp. The IL2 cloned insert is 814 bp. The size of the vector+insert=4975 bp.

First we calculate the mass of a single plasmid molecule. The size of the entire plasmid (plasmid+insert) is used in this calculation using DNA Mass Formula.

m=(n)(1.096×10⁻²¹ g/bp);

m=mass
n=plasmid size (bp)

In the case of IL2:

m=4975 bp(1.096×10⁻²¹)g/bp

m=5.453×10⁻¹⁸ g=mass of a single plasmid molecule.

We then calculate the mass of plasmid containing the copy numbers of interest, in this example 10⁸:

### E.g

Copy number (CN) of interest×mass of single plasmid=mass of plasmid DNA needed for Copy Number of interest

(10*8CN)×(5.453×10⁻¹⁸ g)=5.453e-10 g

Therefore, the mass of plasmid DNA needed for 10*8 copy numbers=5.453e-10 g

We then calculate the concentrations of plasmid DNA needed to achieve the copy numbers of interest (table 2).

TABLE 2

| Copy Number of Interest | Mass of Plasmid DNA (g) |
|---|---|
| 10*8 | 5.453 × 10⁻¹⁰ |
| 10*7 | 5.453 × 10⁻¹¹ |
| 10*6 | 5.453 × 10⁻¹² |
| 10*5 | 5.453 × 10⁻¹³ |
| 10*4 | 5.453 × 10⁻¹⁴ |
| 10*3 | 5.453 × 10⁻¹⁵ |
| 10*2 | 5.453 × 10⁻¹⁶ |
| 10*1 | 5.453 × 10⁻¹⁷ |

We next calculate the concentrations of plasmid DNA needed to achieve the copy numbers of interest, dividing the mass needed for respective copy number of interest by the volume pipetted into each reaction (1.5 μl) see table 3.

TABLE 3

| | Mass of Plasmid | Volume used | Final Conc of |
|---|---|---|---|
| Copy Number | DNA needed | in each | plasmid |
| of Interest | (g) | QRT-PCR μl | DNA(g/μl) |
| 10*9 | 5.453 × 10⁻⁹ | 1.5 | 3.635 × 10⁻⁹ |
| 10*8 | 5.453 × 10⁻¹⁰ | 1.5 | 3.635 × 10⁻¹⁰ |
| 10*7 | 5.453 × 10⁻¹¹ | 1.5 | 3.635 × 10⁻¹¹ |
| 10*6 | 5.453 × 10⁻¹² | 1.5 | 3.635 × 10⁻¹² |
| 10*5 | 5.453 × 10⁻¹³ | 1.5 | 3.635 × 10⁻¹³ |
| 10*4 | 5.453 × 10⁻¹⁴ | 1.5 | 3.635 × 10⁻¹⁴ |
| 10*3 | 5.453 × 10⁻¹⁵ | 1.5 | 3.635 × 10⁻¹⁵ |
| 10*2 | 5.453 × 10⁻¹⁶ | 1.5 | 3.635 × 10⁻¹⁶ |
| 10*1 | 5.453 × 10⁻¹⁷ | 1.5 | 3.635 × 10⁻¹⁷ |

The final step is to prepare a serial dilution of the plasmid DNA. The cloned sequences are highly concentrated in purified plasmid DNA stocks. A series of serial dilutions are performed if necessary to achieve a working stock of plasmid DNA for quantitative RT PCR applications.

Once the plasmid is at a workable concentration, the following formula is used to calculate the volume needed to prepare the 10*8 copy standard dilution. (in case of IL2 dilution III)

C₁V₁=C₂V₂

TABLE 4

| | Source of | Initial | Volume of | | Final | Final | |
|---|---|---|---|---|---|---|---|
| | Plasmid | Conc | plasmid | Vol of | Volume | Conc | Resulting |
| | DNA for | (g/μl) | DNA | Diluent | (μl) | (g/μl) | Copy |
| Dilution | dilution | (C₁) | (μl) (V₁) | (μl) | (V₂) | (C₂) | Numbers |
| I | Stock | 515.7e⁻⁹ | 10 | 990 | 1000 | 5.157e⁻⁹ | N/A |
| II | Dil I | 5.157e⁻⁹ | 70.486 | 29.514 | 100 | 3.635e⁻⁹ | 10*⁹ |
| III | Dil II | 3.635e⁻⁹ | 10 | 90 | 100 | 3.635e⁻¹⁰ | 10*⁸ |
| IV | Dil III | 3.635e⁻¹⁰ | 10 | 90 | 100 | 3.635e⁻¹¹ | 10*⁷ |
| V | Dil IV | 3.635e⁻¹¹ | 10 | 90 | 100 | 3.635e⁻¹² | 10*⁶ |
| VI | Dil V | 3.635e⁻¹² | 10 | 90 | 100 | 3.635e⁻¹³ | 10*⁵ |
| VII | Dil VI | 3.635e⁻¹³ | 10 | 90 | 100 | 3.635e⁻¹⁴ | 10*⁴ |
| VIII | Dil VII | 3.635e⁻¹⁴ | 10 | 90 | 100 | 3.635e⁻¹⁵ | 10*³ |
| IX | Dil VIII | 3.635e⁻¹⁵ | 10 | 90 | 100 | 3.635e⁻¹⁶ | 10*² |
| X | Dil IX | 3.635e⁻¹⁶ | 10 | 90 | 100 | 3.635e⁻¹⁷ | 10*¹ |

The diluent used in these dilutions was sterile TE buffer (10 mM Tris HCL, 1 mM EDTA pH 8.0 with 10 μg/ml double stranded herring DNA (sigma).

Dilutions II to X were used for quantitative PCR application.

### IL7 Standard Curve

The stock of IL7 plasmid DNA was determined to be 3365.6 ng/μl by spectrophotometric analysis. The vector size for pCMV-SPORT6 is 4396 bp. The IL2 cloned insert is 2125 bp. The size of the vector+insert=6521 bp.

First we calculate the mass of a single plasmid molecule. The size of the entire plasmid (plasmid+insert) is used in this calculation using DNA Mass Formula.

m=(n)(1.096×10⁻²¹ g/bp);

m=mass
n=plasmid size (bp)

In the case of IL7:

m=6521 bp(1.096×10⁻²¹) g/bp

m=7.147×10⁻¹⁸ g=mass of a single plasmid molecule.

We then calculate the mass of plasmid containing the copy numbers of interest, in this example 10⁸:

### E.g

Copy number (CN) of interest×mass of single plasmid=mass of plasmid DNA needed for Copy Number of interest

(10*8CN)×(7.147×10⁻¹⁸ g)=7.147e-10 g

Therefore, the mass of plasmid DNA needed for 10*8 copy numbers=7.147e-10 g

We then calculate the concentrations of plasmid DNA needed to achieve the copy numbers of interest (table 5).

TABLE 5

| Copy Number of Interest | Mass of Plasmid DNA (g) |
|---|---|
| 10*8 | 7.147 × 10⁻¹⁰ |
| 10*7 | 7.147 × 10⁻¹¹ |
| 10*6 | 7.147 × 10⁻¹² |
| 10*5 | 7.147 × 10⁻¹³ |
| 10*4 | 7.147 × 10⁻¹⁴ |
| 10*3 | 7.147 × 10⁻¹⁵ |
| 10*2 | 7.147 × 10⁻¹⁶ |
| 10*1 | 7.147 × 10⁻¹⁷ |

We next calculate the concentrations of plasmid DNA needed to achieve the copy numbers of interest, dividing the mass needed for respective copy number of interest by the volume pipetted into each reaction (1.5 μl) see table 6

TABLE 6

| | Mass of Plasmid | Volume used | Final Conc |
|---|---|---|---|
| Copy Number | DNA needed | in each | of plasmid |
| of Interest | (g) | QRT-PCR μl | DNA(g/μl) |
| 10*9 | 7.147 × 10⁻⁹ | 1.5 | 4.765 × 10⁻⁹ |
| 10*8 | 7.147 × 10⁻¹⁰ | 1.5 | 4.765 × 10⁻¹⁰ |
| 10*7 | 7.147 × 10⁻¹¹ | 1.5 | 4.765 × 10⁻¹¹ |
| 10*6 | 7.147 × 10⁻¹² | 1.5 | 4.765 × 10⁻¹² |
| 10*5 | 7.147 × 10⁻¹³ | 1.5 | 4.765 × 10⁻¹³ |
| 10*4 | 7.147 × 10⁻¹⁴ | 1.5 | 4.765 × 10⁻¹⁴ |
| 10*3 | 7.147 × 10⁻¹⁵ | 1.5 | 4.765 × 10⁻¹⁵ |
| 10*2 | 7.147 × 10⁻¹⁶ | 1.5 | 4.765 × 10⁻¹⁶ |
| 10*1 | 7.147 × 10⁻¹⁷ | 1.5 | 4.765 × 10⁻¹⁷ |

The final step is to prepare a serial dilution of the plasmid DNA. The cloned sequences are highly concentrated in purified plasmid DNA stocks. A series of serial dilutions are performed if necessary to achieve a working stock of plasmid DNA for quantitative RT PCR applications. Once the plasmid is at a workable concentration, the following formula is used to calculate the volume needed to prepare the 10*8 copy standard dilution. (table 7)

C₁V₁=C₂V₂

TABLE 7

| | Source of | Initial | Volume of | | Final | Final | |
|---|---|---|---|---|---|---|---|
| | Plasmid | Conc | plasmid | Vol of | Volume | Conc | Resulting |
| | DNA for | (g/μl) | DNA | Diluent | (μl) | (g/μl) | Copy |
| Dilution | dilution | (C₁) | (μl) (V₁) | (μl) | (V₂) | (C₂) | Numbers |
| I | Stock | 33.659e⁻⁷ | 14.156 | 85.844 | 100 | 4.765e⁻⁷ | 10*¹¹ |
| II | Dil I | 4.765e⁻⁷ | 10 | 90 | 100 | 4.765e⁻⁸ | 10*¹⁰ |
| III | Dil II | 4.765e⁻⁸ | 10 | 90 | 100 | 4.765e⁻⁹ | 10*⁹ |
| IV | Dil III | 4.765e⁻⁹ | 10 | 90 | 100 | 4.765e⁻¹⁰ | 10*⁸ |
| V | Dil IV | 4.765e⁻¹⁰ | 10 | 90 | 100 | 4.765e⁻¹¹ | 10*⁷ |
| VI | Dil V | 4.765e⁻¹¹ | 10 | 90 | 100 | 4.765e⁻¹² | 10*⁶ |
| VII | Dil VI | 4.765e⁻¹² | 10 | 90 | 100 | 4.765e⁻¹³ | 10*⁵ |
| VIII | Dil VII | 4.765e⁻¹³ | 10 | 90 | 100 | 4.765e⁻¹⁴ | 10*⁴ |
| IX | Dil VIII | 4.765e⁻¹⁴ | 10 | 90 | 100 | 4.765e⁻¹⁵ | 10*³ |
| X | Dil IX | 4.765e⁻¹⁵ | 10 | 90 | 100 | 4.765e⁻¹⁶ | 10*² |
| XI | Dil X | 4.765e⁻¹⁶ | 10 | 90 | 100 | 4.765e⁻¹⁷ | 10*¹ |

The diluent used in these dilutions was sterile TE buffer (10 mM Tris HCL, 1 mM EDTA pH 8.0 with 10 μg/ml double stranded herring DNA (sigma).

Dilutions IV to XI were used for quantitative PCR application.

### Primers and Probes for IL2 and IL7

All primers and probes for IL2 and IL7 were synthesized at Applied Biosystems (Foster City, Calif.). Both IL2 and IL7 were obtained as a precustomised primer and probe mix. (Taqman 0 Gene Expression Assays ID Hs00174114_ml and for IL7 is Taqman® Gene Expression Assays ID Hs00174202_ml).

Expression of IL2 and IL7 in patient samples were normalised to 10*7 copy numbers of the house-keeping gene β-Actin. The β-Actin primers and probe were designed and customised as per Stordeur at al. (Stordeur et al, 2002).

The probe stock for β-Actin (40 pmol/L) was stored at −20° C. and a working dilution of 4 pmol/L, with 200 nM probe used per 20 μL QRT-PCR reaction. 300 nM of forward and reverse primers were used per 204 QRT-PCR reaction.

Sequences for β-Actin
Forward Primer
(SEQUENCE ID NO: 1)
GGATGCAGAAGGAGATCACTG
Reverse Primer
(SEQUENCE ID NO: 2)
CGATCCACACGGAGTACTTG
Probe
(SEQUENCE ID NO: 3)
6Fam-CCCTGGCACCCAGCACAATG-Tamra-p
Sequences for IL-23
IL23p19
(SEQUENCE ID NO: 4)
F533: TACTGGGCCTCAGCCAACT
(SEQUENCE ID NO: 5)
R649: GAAGGATTTTGAAGCGGAGAA
(SEQUENCE ID NO: 6)
P597: 6Fam-CCTCAGTCCCAGCCAGCCATG-Tamra-p
(As per O'Dwyer et al, Intensive Care
Medicine; 2008, 34, (4), 683-91.)

While the methods section above has been described in relation to quantifying mRNA copy number of IL-2 and IL-7, the same methods may be employed to determine mRNA copy numbers of the other prognostic variables of the present invention.

### BRIEF DESCRIPTION OF THE RESULTS

Pneumonia is a common complication after major surgery in humans and is particularly common after thoracic surgery. Post-operative pneumonia prolongs hospital stay, and may precipitate severe sepsis and septic shock and is associated with an excess mortality rate. The occurrence of pneumonia in the first few days after surgery is an unpredictable event.

In this study of 60 patients after elective thoracic surgery, 19 developed post-operative respiratory tract infection.

Prior to surgery IL-2, IL-6, IL-7, IL-12, IL-23, IL-27, TNF-α and Interferon-γ mRNA were assayed from peripheral blood leukocytes, and the mRNA levels for these cytokines were similar in patients who subsequently developed pneumonia and those who had an uneventful recovery (Table 1).

TABLE 1
Cytokine mRNA levels in peripheral blood leukocytes prior to surgery.

| | Median | 10^{th}-90^{th} centile Range |
|---|---|---|
| IL-2 | 210 | (38-656) |
| (n = 57) | | |
| TGFβ-1 | 2,005,616 | (774613-3732103) |
| (n = 48) | | |
| IL-7 | 1159 | (354-2122) |
| (n = 60) | | |
| IL-10 | 657 | (185-3246) |
| (n = 57) | | |
| IL-12 | 11503 | (4940-47257) |
| (n = 59) | | |
| IL-23 | 44711 | (4707-134827) |
| (n = 60) | | |
| IL-27 | 384 | (29.8-856) |
| (n = 55) | | |
| TNFα | 54984 | (5333-716664) |
| (n = 60) | | |
| INFγ | 659 | (142-2271) |
| (n = 55) | | |

All values are quotes as absolute copy numbers of mRNA per 10 million copy numbers of β-Actin

On the first day after surgery IL-2, IL-7 and IL-23 mRNA levels in peripheral blood leukocytes were less in patients who subsequently developed pneumonia when compared with patients who had an uneventful recovery (Table 2).

TABLE 2
Cytokine mRNA levels in peripheral blood Leukocytes of patients on the
first day after thoracotomy

| | Pneumonia | No Pneumonia | P Value |
|---|---|---|---|
| IL-2 | 65 (14-216) | 129 (31-508) | 0.03 |
| N | 14 | 37 | |
| TGFβ-1 | 1.7*10⁶ | 1.5*10⁶ | ns |
| N | (0.7*10⁶-3.5*10⁶) | (0.57*10⁶-3.8*10⁶) | |
| | 14 | 33 | |
| IL-7 | 727 (31-1804) | 1068 (402-2319) | 0.06 |
| N | 19 | 41 | |
| IL-10 | 3262 (482-17715) | 1854 (230-10389) | 0.16 |
| N | 17 | 38 | |
| IL-12 | 10924 (5075-31154) | 11503 (6838-28321) | ns |
| N | 19 | 41 | |
| IL-23 | 15465 (8071-51614) | 31420 (7101-206943) | 0.02 |
| N | 18 | 38 | |
| IL-27 | 293 (101-625) | 310 (30-1226) | ns |
| N | 17 | 40 | |
| TNFα | 41162 (24749-189465) | 42922 (24199-302944) | ns |
| N | 18 | 41 | |
| IFNγ | 329 (29-1603) | 336 (123-3884) | ns |
| N | 18 | 40 | |

All values are quoted as absolute copy numbers of mRNA per 10 million copy numbers of β-Actin. All values are quotes as median and 10^{th} to 90^{th} centile range. Comparison is by Wilcoxon Rank Sum test.

On the fifth day after surgery IL-2, IL-7 and Interferon-γ mRNA levels in peripheral blood leukocytes were less in patients who subsequently developed pneumonia when compared with patients who had an uneventful recovery (Table 3).

TABLE 3
Cytokine mRNA levels in peripheral blood leukocytes of patients on the
fifth day after thoracotomy.

| | Pneumonia | No Pneumonia | P Value |
|---|---|---|---|
| IL-2 | 85 (15-372) | 117 (24-247) | ns |
| N | 15 | 33 | |
| IL-7 | 785 (144-1676) | 1485 (431-3651) | 0.005 |
| N | 17 | 39 | |
| IL-10 | 1866 (229-5656) | 1232 (77-2987) | ns |
| N | 15 | 37 | |
| IL-12 | 8966 (4401-42464) | 8778 (3313-40995) | ns |
| N | 17 | 37 | |
| IL-23 | 31812 (3703-1068548) | 37488 (4486-176848) | ns |
| N | 15 | 39 | |
| IL-27 | 281 (78-1155) | 253 (33-667) | ns |
| N | 14 | 37 | |
| TNFα | 80537 (12942-431292) | 58116 (8861-793068) | ns |
| N | 17 | 38 | |
| IFNγ | 305 (102-1522) | 640 (144-1820) | 0.03 |
| N | 15 | 35 | |

All values are quotes as absolute copy numbers of mRNA per 10 million copy numbers of β-Actin. All values are quotes as median and 10^{th} to 90^{th} centile range. Comparison is by Wilcoxon Rank Sum test.

When cytokine mRNA levels on the first post operative day were compared to preoperative values, patients who developed pneumonia had greater reduction in IL-2, IL-7, IL-23 and TNF-α, with a borderline significantly reduction in IL-27 mRNA (Table 4)

TABLE 4
Relative Change in cytokine mRNA in the first 24 hours after surgery

| | Pneumonia | No Pneumonia | P Value |
|---|---|---|---|
| IL-2 | 6.7 (2.1-28) | 1.1 (0.3-4.7) | <0.0001 |
| N | 14 | 37 | |
| IL-7 | 1.28 (0.88-6.23) | 0.92 (0.52-2.1) | 0.003 |
| N | 18 | 41 | |
| IL-10 | 0.31 (0.06-1.14) | 0.36 (0.09-1.69) | ns |
| N | 17 | 36 | |
| IL-12 | 1.3 (0.48-32.3) | 1.24 (0.24-7.6) | ns |
| N | 18 | 38 | |
| IL-23 | 2.84 (1.39-7.95) | 0.83 (0.10-6.92) | 0.0006 |
| N | 18 | 39 | |
| IL-27 | 1.15 (0.4-3.29) | 0.85 (0.2-4.15) | 0.05 |
| N | 16 | 36 | |
| TNFα | 1.63 (0.24-18.6) | 0.79 (0.15-3.67) | 0.004 |
| N | 18 | 41 | |
| IFNγ | 1.94 (0.74-22) | 1.4 (0.12-3.12) | ns |
| N | 16 | 37 | |

Data represents the ratio of pre operative mRNA to post operative mRNA; the preoperative value divided by the post operative value. All values are quotes as median and 10^{th} to 90^{th} centile range. Comparison is by Wilcoxon Rank Sum test.

When cytokine mRNA levels on the fifth post-operative day were compared with preoperative values, the reduction in Interferon-γ, IL-2 and IL-7 was greater in patients who developed pneumonia than those who had an uneventful recovery (Table 5).

TABLE 5
Relative Change in cytokine mRNA in 5 days after surgery

| | Pneumonia | No Pneumonia | P Value |
|---|---|---|---|
| IL-2 | 3.7 (0.72-13.5) | 1.4 (0.44-8.2) | 0.02 |
| N | 15 | 33 | |
| IL-7 | 1.48 (0.73-3.6) | 0.71 (0.34-1.6) | 0.0001 |
| N | 17 | 38 | |
| IFNγ | 2.4 (0.4-6.5) | 1.04 (0.3-8) | 0.05 |
| N | 13 | 32 | |

Data represents the ratio of pre operative mRNA to post operative mRNA; the preoperative value divided by the post operative value. All values are quotes as median and 10^{th} to 90^{th} centile range. Comparison is by Wilcoxon Rank Sum test.

An algorithm based on absolute values of cytokine mRNA was developed from assays of cytokine mRNA on the first post-operative day, using recursive partitioning. Recursive partitioning, a multivariate method of data analysis, was used to identify distinct patterns of cytokine mRNA on an objective basis.

Models of Cytokine mRNA and the Occurrence of Pneumonia

It would be very beneficial to be able to predict the occurrence of pneumonia after surgery. However there were no cytokine mRNA assays from the preoperative testing which were predictive of subsequent pneumonia.

Several algorithms, based on cytokine mRNA assays on the first day after surgery, were predictive of subsequent pneumonia. These algorithms were based on combinations of IL-2, IL-23, IL-7, IL-10, TNF and interferon g mRNA.

Algorithm Based on Absolute Values of IL-23 mRNA from the First Day after Surgery

In this algorithm patients with IL-23 mRNA copy numbers greater than or equal to 60684.9 are regarded as having a low risk of pneumonia. Patients with lesser IL-23 mRNA copy have a higher risk of pneumonia. Using this algorithm none of 13 patients with low risk developed pneumonia, while 17 of 44 patients with greater risk developed pneumonia. The Chi square of this model is 17 and a p value of 0.0001.

Algorithm Based on Absolute Values of IL-23 and IL-7 mRNA from the First Day after Surgery

In this algorithm patients with IL-23 mRNA copy numbers greater than or equal to 60684.9 are regarded as having a low risk of pneumonia. Patients with lesser IL-23 mRNA copy numbers and with IL-7 mRNA copy numbers less than 582.9 have a high risk of pneumonia, while the remaining patients have an intermediate risk of developing pneumonia. Using this algorithm none of 13 patients with low risk developed pneumonia, while 9 of 33 patients with intermediate risk developed pneumonia, and 8 of 11 patients with high risk developed pneumonia. The Chi square of this model is 17 and a p value of 0.0001.

Algorithm Based on Absolute Values of Il-23 and Interferon-γ from the First Day after Surgery

In this algorithm patients with IL-23 mRNA copy numbers greater than or equal to 60684.9 are regarded as having a low risk of pneumonia. Patients with lesser IL-23 mRNA copy numbers and with Interferon-γ mRNA copy numbers less than 101 have a high risk of pneumonia, while the remaining patients have an intermediate risk of developing pneumonia.

Using this algorithm none of 13 patients with low risk developed pneumonia, while 12 of 37 patients with intermediate risk developed pneumonia, and 5 of 6 patients with high risk developed pneumonia. The Chi square of this model is 16.7 and a p value of 0.0001.

Algorithm Based on Absolute Values of IL-23 and TNFa from the First Day after Surgery

In this algorithm patients with IL-23 mRNA copy numbers greater than or equal to 60684.9 are regarded as having a low risk of pneumonia. Patients with lesser IL-23 mRNA copy numbers and with TNF-α mRNA copy numbers greater than 184990 have a high risk of pneumonia, while the remaining patients have an intermediate risk of developing pneumonia.

Using this algorithm none of 13 patients with low risk developed pneumonia, while 13 of 37 patients with intermediate risk developed pneumonia, and 5 of 7 patients with high risk developed pneumonia. The Chi square of this model is 14 and a p value of 0.0006.

Algorithm Based on IL-23/IL-27 Ratio and Interferon-γ/IL-10 Ratio from the First Day after Surgery

It is known from prior research that the combination of IL-23 to Il-27 ratio and Interferon g to IL-10 ratio may be predictive of outcome in patients with sepsis. In this algorithm, patients with IL-23/IL-27 ratio of mRNA copy numbers greater than or equal to 251 are regarded as having a low risk of pneumonia. Patients with lesser IL-23/IL-27 ratio of mRNA copy numbers and with Interferon-γ/IL-10 ratio of mRNA copy numbers less than 0.0171 have a high risk of pneumonia, while the remaining patients have an intermediate risk of developing pneumonia. Using this algorithm none of 14 patients with low risk developed pneumonia, while 12 of 33 patients with intermediate risk developed pneumonia, and 4 of 6 patients with high risk developed pneumonia. The Chi square of this model is 14 and a p value of 0.0009.

### Algorithm Based on Relative Change in IL-23

In patients who developed pneumonia, IL-23 decreased after surgery. This reduction can be expressed as a ratio, where the preoperative value is divided by the post operative value, with ratios greater than 1 representing a reduction. In this algorithm, patients with an IL-23 ratio less than 1.207 are at low risk of developing pneumonia, while in the patients with IL-23 ratio greater than this, are at high risk of developing. Using this algorithm none of the 25 low risk patients developed pneumonia, while 18 of 32 high risk patients developed pneumonia. The Chi square of this model is 27 and the p value is <0.0001

Algorithm Based on Relative Change in IL-23 and IL-7 in the First Day after Surgery

In patients who developed pneumonia, both IL-23 and IL-7 decreased after surgery. This reduction can be expressed as a ratio, where the preoperative value is divided by the post operative value, with ratios greater than 1 representing a reduction. In this algorithm, patients with an IL-23 ratio less than 1.207 are at low risk of developing pneumonia, while in the patients with Il-23 ratio greater than this; those with an IL-7 ratio greater than 1.106 are at high risk of developing pneumonia with the remaining patients representing an intermediate risk group. Using this algorithm none of the 25 low risk patients developed pneumonia, while 2 of 7 intermediate risk patients developed pneumonia, and 16 of 25 high risk patients developed pneumonia. The Chi square of this model is 30 and the p value is <0.0001

Algorithm Based on Relative Change in IL-23 and Interferon-γ in the First Day after Surgery

In patients who developed pneumonia, both IL-23 and Interferon-γ decreased after surgery. This reduction can be expressed as a ratio, where the preoperative value is divided by the post operative value, with ratios greater than 1 representing a reduction. In this algorithm, patients with an IL-23 ratio less than 1.207 are at low risk of developing pneumonia, while in the patients with IL-23 ratio greater than this, those with an interferon ratio greater than 1.106 are at high risk of developing pneumonia with the remaining patients representing an intermediate risk group. Using this algorithm none of the 25 low risk patients developed pneumonia, while 2 of 7 intermediate risk patients developed pneumonia, and 16 of 25 high risk patients developed pneumonia. The Chi square of this model is 30 and the p value is <0.0001.

Algorithm Based on Relative Change in IL-2 on the First Day after Surgery

In patients who developed pneumonia, IL-2 decreased after surgery. This reduction can be expressed as a ratio, where the preoperative value is divided by the post-operative value, with ratios greater than 1 representing a reduction. In this algorithm, patients with an IL-2 ratio less than 1.5192 are at low risk of developing pneumonia, while in the patients with IL-2 ratio greater than this, are at high risk of developing. Using this algorithm none of the 25 low risk patients developed pneumonia, while 14 of 26 high risk patients developed pneumonia. The Chi square of this model is 22 and the p value is <0.0001.

Algorithm Based on Relative Change in IL-2 and IL-23 on the First Day after Surgery

In patients who developed pneumonia, both IL-23 and IL-2 decreased after surgery. This reduction can be expressed as a ratio, where the preoperative value is divided by the post-operative value, with ratios greater than 1 representing a reduction. In this algorithm, patients with an IL-2 ratio less than 1.5198 are at low risk of developing pneumonia, while in the patients with IL-2 ratio greater than this, those with an IL-23 ratio greater than 1.4115 are at high risk of developing pneumonia with the remaining patients representing an intermediate risk group. Using this algorithm none of the 25 low risk patients developed pneumonia, while none of 7 intermediate risk patients developed pneumonia, and 14 of 18 high risk patients developed pneumonia. The Chi square of this model is 40 and the p value is <0.0001.

### Algorithm Based on Change in IL-2 Ct Value Between Pre and Post Operative Patient—Delta Ct

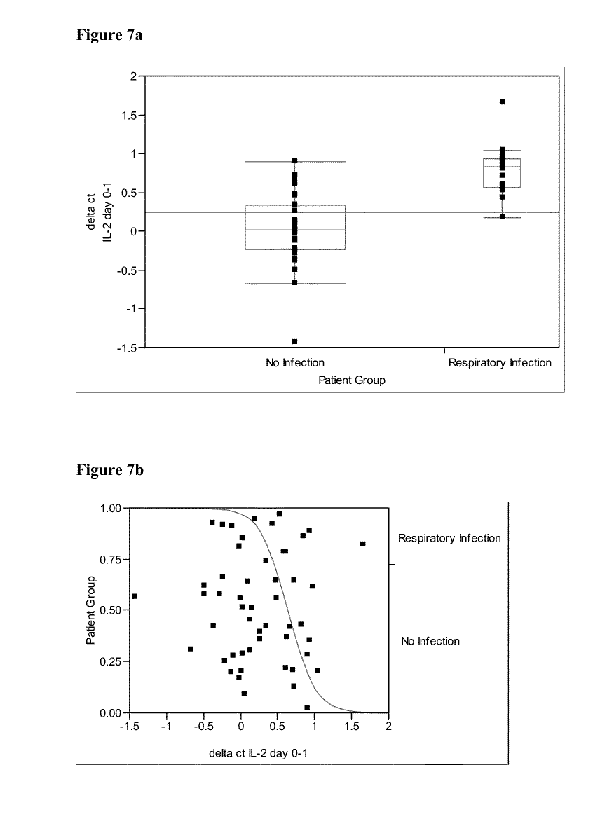

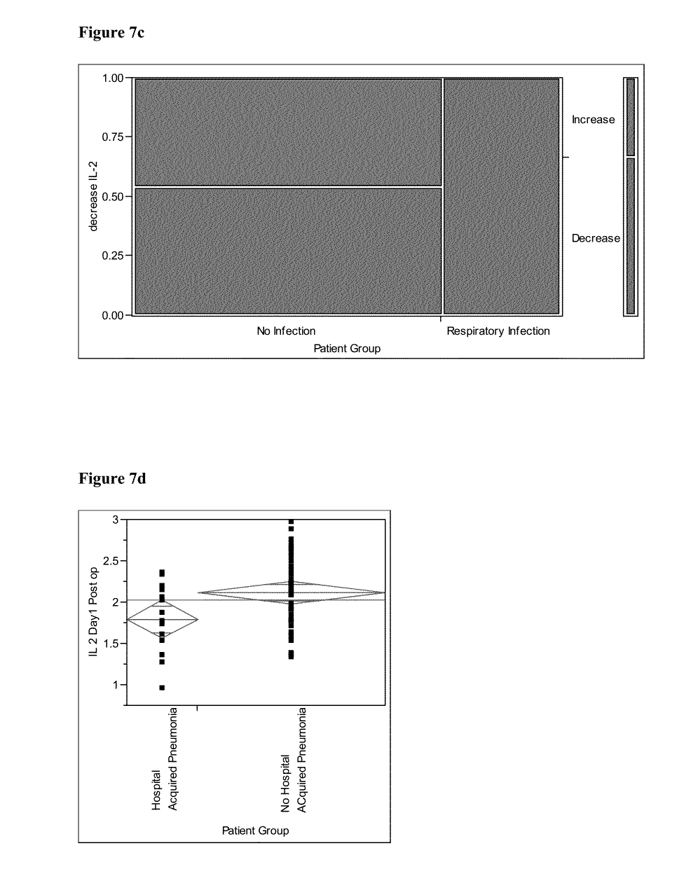

An alternate way of analysing data is to examine the difference in RT-PCR Ct value from pre operative to post operative. In this data the normalised Ct on post operative day 1 is subtracted from the normalised Ct of the pre operative sample. This is another way of stating the ratio of post operative cytokine mRNA to pre operative cytokine mRNA. This difference can be referred to as a delta Ct. In these results positive values of delta Ct reflect a decrease in cytokine mRNA: a positive Ct Value for IL-2 reflects an increased risk of respiratory tract infection whereas a negative value for delta Ct represents low risk of infection (FIGS. 7a, 7b and 7c).

### Algorithm Based on Change in IL-7 Ct Value Between Pre and Post Operative Patients

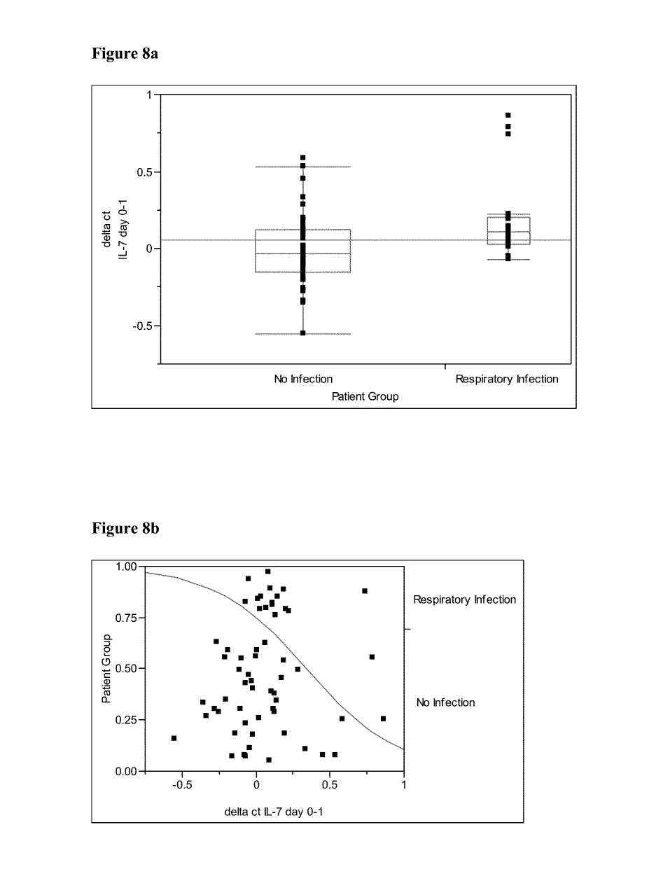

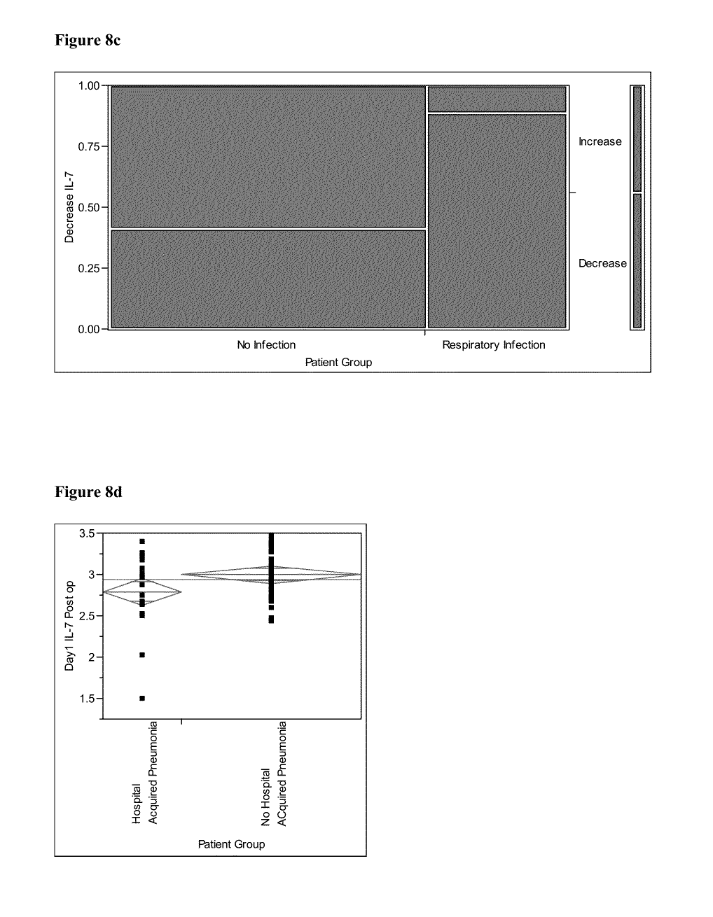

An alternate way of analysing data is to examine the difference in RT-PCR Ct value from pre operative to post operative. In this data the normalised Ct on post operative day 1 is subtracted from the normalised Ct of the pre operative sample. This is another way of stating the ratio of post operative cytokine mRNA to pre operative cytokine mRNA. This difference can be referred to as a delta Ct. In these results positive values of delta Ct reflect a decrease in cytokine mRNA, a positive Ct Value for IL-7 reflects an increased risk of respiratory tract infection (FIGS. 8a, 8b and 8c).

### Algorithm Based on Change in IL-23 Ct Value Between Pre and Post Operative Patients

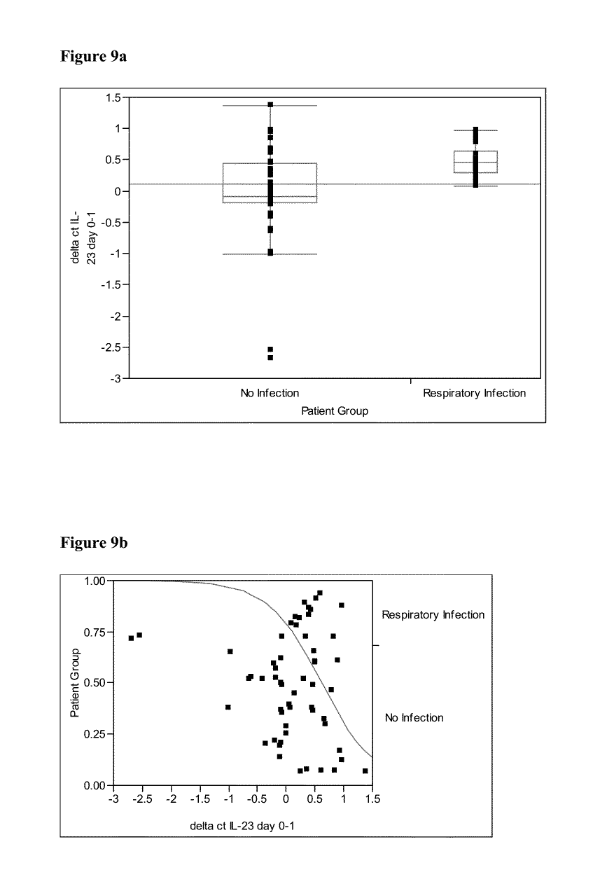

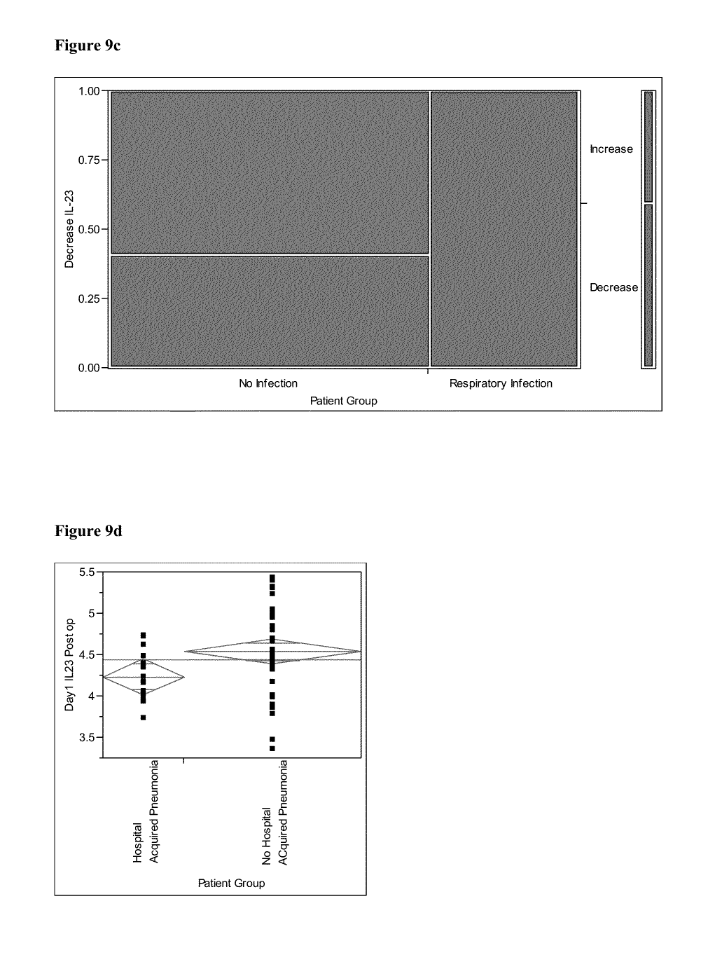

An alternate way of analysing data is to examine the difference in RT-PCR Ct value from pre operative to post operative. In this data the normalised Ct on post operative day 1 is subtracted from the normalised Ct of the pre operative sample. This is another way of stating the ratio of post operative cytokine mRNA to pre operative cytokine mRNA. This difference can be referred to as a delta Ct. In these results positive values of delta Ct reflect a decrease in cytokine mRNA, a positive Ct Value for IL-23 reflects an increased risk of respiratory tract infection (FIGS. 9a, 9b and 9c).

### Algorithm Based on Change in TNFα Ct Value Between Pre and Post Operative Patients

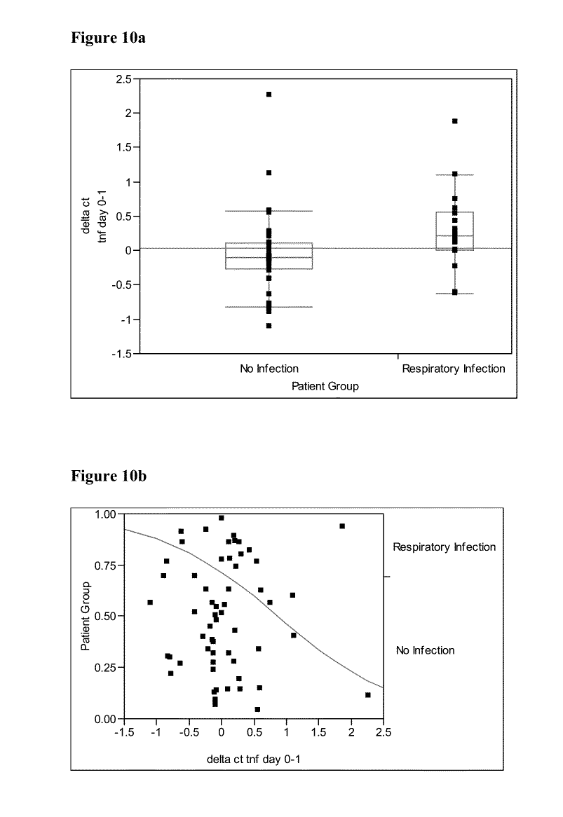

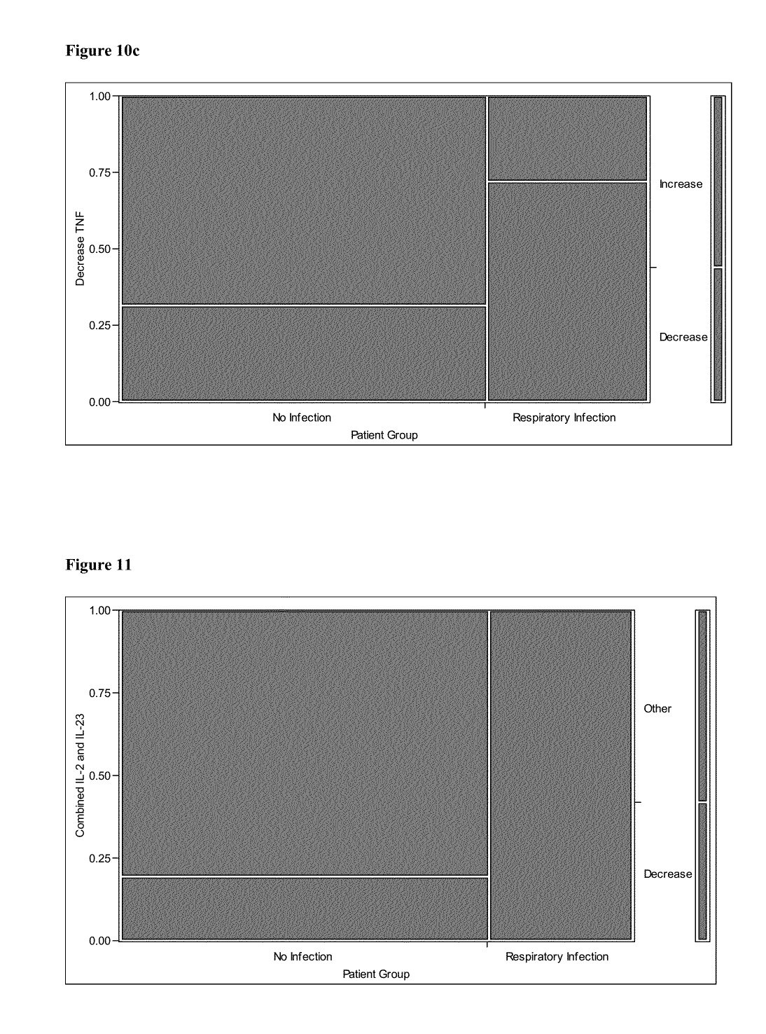

An alternate way of analysing data is to examine the difference in RT-PCR Ct value from pre operative to post operative. In this data the normalised Ct on post operative day 1 is subtracted from the normalised Ct of the pre operative sample. This is another way of stating the ratio of post operative cytokine mRNA to pre operative cytokine mRNA. This difference can be referred to as a delta Ct. In these results positive values of delta Ct reflect a decrease in cytokine mRNA, a positive Ct Value for TNFα reflects a risk of respiratory tract infection (FIGS. 10a, 10b and 10c).

Algorithm Based on Absolute Post-Operative IL-2 mRNA Copy Number Values

The occurrence of post operative pneumonia correlates with gene transcription data for single cytokines. In this analysis IL-2 is expressed as a corrected Log base 10 copy number. Thus IL-2 mRNA copy number values are lower on the first day after surgery in patients who subsequently develop post operative pneumonia (FIG. 7d).

Algorithm Based on Absolute Post-Operative IL-7 mRNA Copy Number Values

The occurrence of post operative pneumonia correlates with gene transcription data for single cytokines. In this analysis IL-2 is expressed as a corrected Log base 10 copy number. Thus IL-7 mRNA copy number values are lower on the first day after surgery in patients who subsequently develop post operative pneumonia (FIG. 8d).

Algorithm Based on Absolute Post-Operative IL-23 mRNA Copy Number Values

The occurrence of post operative pneumonia correlates with gene transcription data for single cytokines. In this analysis IL-2 is expressed as a corrected Log base 10 copy number. Thus IL-23 mRNA copy number values are lower on the first day after surgery in patients who subsequently develop post operative pneumonia (FIG. 9d).

### Algorithm Based on Change in Combined IL-2 and IL23 Ct Value

Powerful discrimination between patients who develop respiratory tract infection is obtained with combined categorical decrease in IL-2 and IL-23 mRNA copy number values. In this analysis 14 of 21 patients with combined decrease in IL-2 and IL-23 mRNA copy number values developed respiratory tract infection, where as none of the other 29 patients developed respiratory tract infection after surgery. Thus patients with a combined decrease in IL2 and IL23 mRNA copy number values have a 5 fold relative risk of developing respiratory tract infection (FIG. 11).

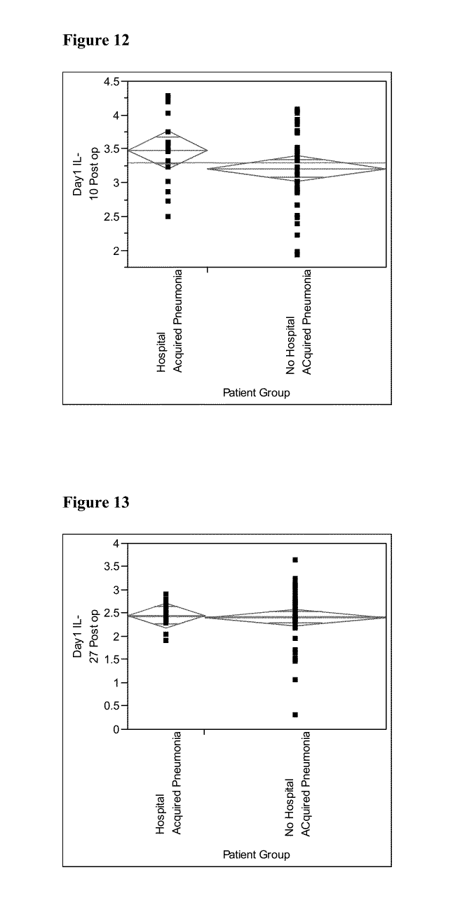

IL-10 mRNA values are similar on the first day after surgery in patients who subsequently develop post operative pneumonia and patients who do not subsequently develop pneumonia. In this analysis IL-10 is expressed as a corrected Log base 10 copy number. As IL-10 is similar in patients who develop pneumonia and those who do not develop pneumonia and thus IL-10 serves as a useful reference cytokine in subsequent algorithms (FIG. 12).

Likewise, IL-27 mRNA values are similar on the first day after surgery in patients who subsequently develop post operative pneumonia and patients who do not subsequently develop pneumonia. In this analysis IL-27 is expressed as a corrected Log base 10 copy number. As IL-27 is similar in patients who develop pneumonia and those who do not develop pneumonia and thus IL-27 serves as a useful reference cytokine in subsequent algorithms (FIG. 13).

### Algorithm Based on Change in IL2, IL7 and IL-23 Relative to IL-10 and IL-27

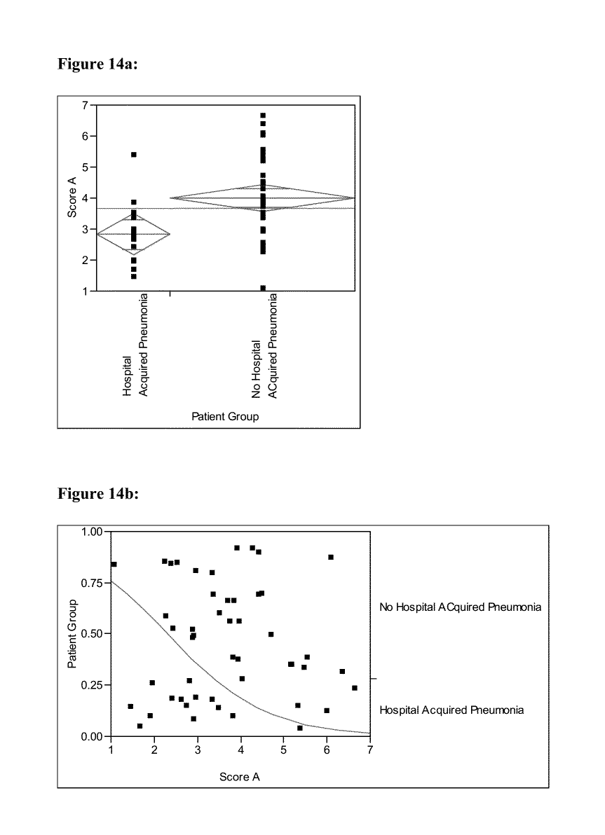

These cytokines, IL-2, IL-7, IL-10, IL-23 and IL-27, can be summated into a combined score by adding the Log base 10 corrected copy numbers of IL-2, IL-7 and IL-23, and subtracting the log base 10 corrected copy number for IL-10 and IL-27. The Score, Score A, which results for this combination of cytokines is significantly lower in patients who subsequently develop pneumonia. In this analysis increasing score, is associated with a decrease in risk for developing post operative pneumonia. Thus for every unit increase in score the risk of developing pneumonia is multiplied by 0.4165, and the risk of developing pneumonia over the range of the score decreases as the score increases, with the highest score associated with a 0.007 times risk of developing pneumonia compared with the lowest score. Conversely for each unit decrease in score the risk of pneumonia increases by 2.4, with a cumulative increased risk from highest to lowest score of 131 fold. (FIGS. 14a and 14b)

### Algorithm Based on Change in IL2 Relative to IL-10

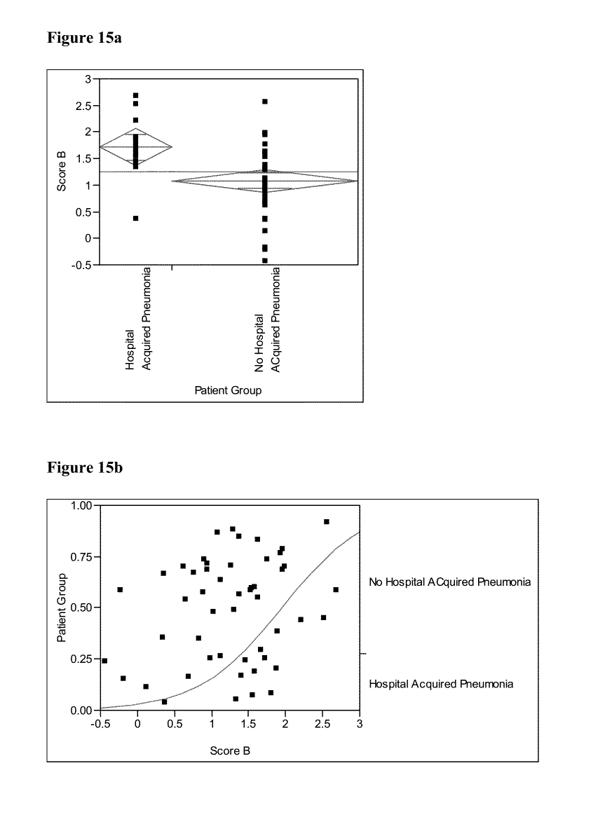

An alternative algorithm involves the difference in log base 10 corrected copy numbers of IL-10 minus those for IL-2. This score is higher in patients who develop pneumonia after surgery. In this analysis increasing score, in this case score B is associated with an increase in risk for developing post operative pneumonia. Thus for every unit increase in score the risk of developing pneumonia is multiplied by 5.96, and the risk of developing pneumonia over the range of the score decreases as the score increases, with the highest score associated with a 263 times risk of developing pneumonia compared with the lowest score. (FIGS. 15a and 15b)

### Algorithm Based on Change in IL2 Relative to IL-10 and IL-27

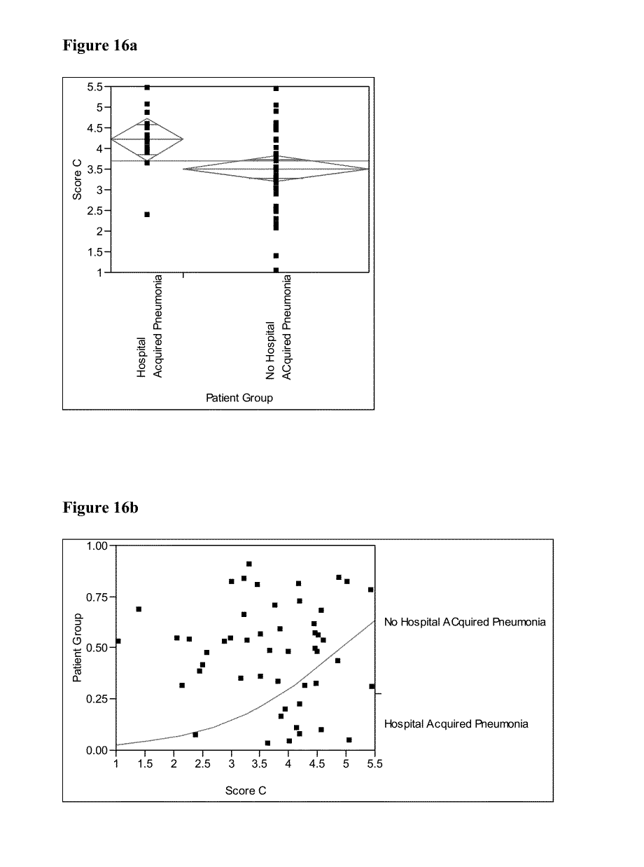

An alternative and algorithm involves the difference in log base 10 corrected copy numbers of IL-10 plus those for IL-27 minus those for IL-2. This score is higher in patients who develop pneumonia after surgery. In this analysis increasing score, in this case score C is associated with a decrease in risk for developing post operative pneumonia. Thus for every unit increase in score the risk of developing pneumonia is multiplied by 2.5, and the risk of developing pneumonia over the range of the score decreases as the score increases, with the highest score associated with a 59.5 times risk of developing pneumonia compared with the lowest score. (FIGS. 16a and 16b)

### Algorithm Based on Change in IL2 Relative to IL-27

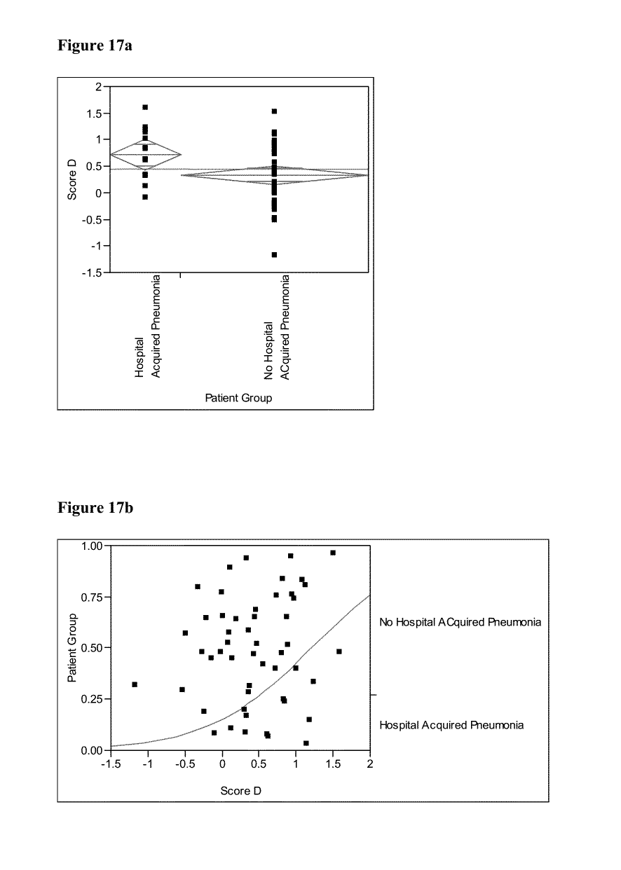

An alternative and algorithm involves the difference in log base 10 corrected copy numbers of IL-27 minus those for IL-2. This score is higher in patients who develop pneumonia after surgery. In this analysis increasing score, in this case score D is associated with a decrease in risk for developing post operative pneumonia. Thus for every unit increase in score the risk of developing pneumonia is multiplied by 4.2, and the risk of developing pneumonia over the range of the score decreases as the score increases, with the highest score associated with a 53 times risk of developing pneumonia compared with the lowest score. (FIGS. 17a and 17b)

The invention is not limited to the embodiment hereinbefore described which may be varied in construction and detail without departing from the spirit of the invention.

1. A method of estimating risk of a patient developing a respiratory tract infection following major surgery, the method comprising the steps:
performing quantitative PCR on a biological sample obtained from the patient within 24 hours of the surgery to measure an absolute mRNA copy number value for a cytokine selected from the group consisting of IL-2, IL-7, IL-23 and combinations thereof;
converting the measured absolute mRNA copy number value to a relative value by
(a) providing a function of the measured absolute mRNA copy number value and a pre-operative reference mRNA copy number value for the same cytokine; or
(b) providing a function of the measured absolute mRNA copy number value and a post-operative mRNA copy number value for a cytokine selected from IL-10 and IL-27; and
correlating the relative value with risk of infection.

2. The method as claimed in claim 1 in which the relative value is a function of the measured absolute mRNA copy number value and a post-operative mRNA copy number value for a cytokine selected from IL-10, IL-27 and combinations thereof, in which the measured absolute mRNA copy number value is selected from IL-2 or the sum of IL-2, IL-7 and IL-23.

3. The method as claimed in claim 1 in which the post-operative mRNA copy number value for a cytokine selected from IL-10, IL-27 and combinations thereof is the mRNA copy number value for IL-10, IL-27 or the sum of IL-10 and IL-27.

4. The method as claimed in claim 1, in which the relative value is calculated using an algorithm selected from the group consisting of:
(IL-2+IL-7+IL-23)−(IL-10+IL-27);
(IL-10+IL-27)−(IL-2+IL-7+IL-23);
(IL-10−IL-2);
(IL-2−IL-10);
(IL-10+IL-27)−IL-2;
IL-2−(IL-10+IL-27);
(IL-27−IL-2); or
(IL-2−IL-27).

5. The method as claimed in claim 4 in which the algorithm is (IL-2+IL-7+IL-23)−(IL-10+IL-27), and wherein the relative value obtained is correlated with risk of infection by comparison with FIG. 14A, 14B or a scale of 1 to 7, or any other equivalent scale.

6. The method as claimed in claim 4 in which the algorithm (IL-10−IL-2) and wherein the relative value obtained is correlated with risk of infection by comparison with FIG. 15A or 15B, or a scale of −0.5 to 3, or any other equivalent scale.

7. The method as claimed in claim 4 in which the algorithm is ((IL-10+IL-27)−IL-2) and wherein the relative value obtained is correlated with risk of infection by comparison with FIG. 16A or 16B, or a scale of 1.0 to 5.5, or any other equivalent scale.

8. The method as claimed in claim 4 in which the algorithm is (IL-27−IL-2) and wherein the relative value obtained is correlated with risk of infection by comparison with FIG. 17A or 17B, or a scale of −1.2 to 2.0, or any other equivalent scale.

9. The method as claimed in claim 1 in which relative value for a cytokine is a function of the pre-operative mRNA copy number value to the post-operative mRNA copy number for that cytokine, and wherein the relative value is correlated with risk of infection by comparison with a reference cut-off value for that cytokine.

10. The method as claimed in claim 9 wherein the relative value is calculated by subtracting the Log 10 of the post-operative mRNA copy number from the Log 10 of the pre-operative mRNA copy number, and in which the reference cut-off value is 0, and wherein a positive number correlates with high risk of infection and a negative number correlates with low risk of infection.

11. The method as claimed in claim 9 wherein the relative value for IL-2 is calculated by providing a ratio of the pre-operative IL-2 mRNA copy number and the post-operative IL-2 mRNA copy number, wherein a ratio of greater than 1.5192 correlates with a high risk of infection, and wherein a ratio of less than 1.5192 correlates with a lower risk of infection.

12. The method as claimed in claim 9 wherein the relative value for IL-23 is calculated by providing a ratio of the pre-operative IL-23 mRNA copy number and the post-operative IL-23 mRNA copy number, wherein a ratio of greater than 1.207 correlates with a high risk of infection, and wherein a ratio of less than 1.207 correlates with a lower risk of infection.

13. The method as claimed in claim 1, in which the risk of infection is estimated using two or more IL-2, IL-7, IL-23, IL-10, IL-27, TNF-α, and Interferon-γ.

14. The method as claimed in claim 13 in which risk of infection is estimated using pre-operative and post-operative IL-23 mRNA copy number values, and wherein the estimated risk of infection is further focussed using pre-operative and post-operative of a cytokine selected from the group consisting of Interferon-γ and combinations thereof.

15. The method as claimed in claim 13 in which risk of infection is estimated using pre-operative and post-operative IL-2 mRNA copy number values, and wherein the estimated risk of infection is further focussed using pre-operative and post-operative of IL-23 copy number values.

16. A method of estimating risk of a patient developing a respiratory tract infection following major surgery, the method comprising the steps:
performing quantitative PCR on a peripheral blood mononuclear cell preparation obtained from the patient before surgery (pre-operative), and within 24 hours following the surgery (post-operative), to measure a pre-operative Ct value and a post-operative Ct value for a cytokine selected from the group consisting of IL-2, IL-23 and combinations thereof and
subtracting the post-operative Ct value from the pre-operative Ct value to provide ΔCt value, wherein a positive ΔCt correlates with a risk of high risk of infection, and a negative value for ΔCt correlates with a low risk of infection.

17. A method of estimating risk of a patient developing a respiratory tract infection following major surgery, the method comprising the steps:
performing quantitative PCR on a peripheral blood mononuclear cell preparation obtained from the patient before surgery (pre-operative), and within 24 hours following the surgery (post-operative), to measure a pre-operative Ct value and a post-operative Ct value for a cytokine selected from the group consisting of IL-2, IL-23 and combinations thereof; and
providing a ratio of the pre-operative mRNA copy number and the post-operative mRNA copy number,
wherein when the cytokine is IL-2, a ratio of greater than 1.5192 correlates with a high risk of infection, or wherein when the cytokine is IL-23, a ratio of greater than 1.207 correlates with a high risk of infection.

18. A method of estimating risk of a patient developing a respiratory tract infection following major surgery, the method comprising the steps:
performing quantitative PCR on a peripheral blood mononuclear cell preparation obtained from the patient within 24 hours following the surgery to measure a post-operative absolute mRNA copy number value for a cytokine selected from the group consisting of IL-2, IL-7, IL-23 and combinations thereof (absolute value);
performing quantitative PCR on a peripheral blood mononuclear cell preparation obtained from the patient within 24 hours following the surgery to measure a post-operative mRNA copy number value for a cytokine selected from the group consisting of IL-10, IL-27 and combinations thereof (reference value);
converting the absolute value to a relative value by providing a function of the absolute value and the reference value; and
correlating the relative value with risk of infection.

19. The method as claimed in claim 18 in which the relative value is calculated by an algorithm selected from the group consisting of:
(IL-2+IL-7+IL-23)−(IL-10+IL-27);
(IL-10+IL-27)−(IL-2+IL-7+IL-23);
(IL-10−IL-2);
(IL-2−IL-10);
(IL-10+IL-27)−IL-2;
IL-2−(IL-10+IL-27);
(IL-27−IL-2); or
(IL-2−IL-27).

20. A method of estimating risk of a patient developing a respiratory tract infection following major surgery, the method comprising the steps performing quantitative PCR on a biological sample obtained from the patient within 24 hours of the surgery to measure an absolute mRNA copy number value for a cytokine selected from the group consisting of IL-2, IL-7, IL-23 and combinations thereof, wherein a decrease in absolute mRNA copy number following surgery correlates with risk of infection.

21. The method as claimed in claim 20 in which the cytokine is IL-2, wherein a post-operative IL-2 mRNA copy number of greater than or equal to 237 (per 10 million copies of beta-actin) correlates with low risk of respiratory tract infection, or wherein a postoperative IL-2 mRNA copy number of less than 237 (per 10 million copies of beta-actin) correlates with a higher risk of respiratory tract infection.

22. The method as claimed in claim 20 in which the cytokine is IL-7, wherein a post-operative IL-7 mRNA copy number of greater than or equal to 476 (per 10 million copies of beta-actin) correlates with low risk of respiratory tract infection, or wherein a post-operative IL-7 mRNA copy number of less than 476 (per 10 million copies of beta-actin) correlates with a higher risk of respiratory tract infection.

23. The method as claimed in claim 20 in which the cytokine is IL-23, wherein a post-operative IL-23 mRNA copy number of greater than or equal to 60684.9 (per 10 million copies of beta-actin) correlates with low risk of respiratory tract infection, or wherein a postoperative IL-23 mRNA copy number of less than 60684.9 (per 10 million copies of beta-actin) correlates with a higher risk of respiratory tract infection.

24. The method as claimed in claim 20 which the risk of infection is further focussed by repeating the method using a second, different, cytokine selected from the group consisting of IL-2, IL-7, IL-23, IL-10, IL-27, TNF-α, and Interferon-γ.

25. The method as claimed in claim 24 in which a patient identified as having a higher risk of respiratory tract infection is further stratified according to (a) post-operative IL-7 mRNA copy number, wherein a IL-7 mRNA copy number of less than 582.9 correlates with a high risk of infection, or
(b) post-operative Interferon-γ mRNA copy number, wherein an Interferon-γ mRNA copy number of less than 101 correlates with a high risk of infection, or (c) post-operative TNF-α mRNA copy number, whereing a TNF-α mRNA copy number of greater than 184990 correlates with a high risk of infection.

26. (canceled)

27. (canceled)

28. The method as claimed in claim 1 in which the biological sample is a peripheral blood mononuclear cell preparation or a mononuclear cell preparation from the buffy coat layer of peripheral blood.

29. (canceled)

30. The method as claimed in claim 1 which is (a) a method of estimating risk of a patient developing respiratory tract infection following cardiothoracic surgery or (b) a method of estimating risk of a patient developing pneumonia following cardiothoracic surgery.

31. (canceled)


SEQUENCE LISTING (6 sequences)

SEQ ID NO: 1 - DNA, 21 bp, Artificial sequence
  Forward primer
ggatgcagaaggagatcactg

SEQ ID NO: 2 - DNA, 20 bp, Artificial sequence
  Reverse primer
cgatccacacggagtacttg

SEQ ID NO: 3 - DNA, 20 bp, Artificial sequence
  Beta-actin probe
ccctggcacccagcacaatg

SEQ ID NO: 4 - DNA, 19 bp, Artificial sequence
  Forward primer
tactgggcctcagccaact

SEQ ID NO: 5 - DNA, 21 bp, Artificial sequence
  Reverse primer
gaaggattttgaagcggagaa

SEQ ID NO: 6 - DNA, 21 bp, Artificial sequence
  IL-23 probe
cctcagtcccagccagccatg